(19) United States
(12) Patent Application Publication
(10) Pub. No.: US 2023/0371902 A1
(43) Pub. Date: Nov. 23, 2023

(54) SHUNT SENSOR IMPLANT DEVICES

(71) Applicant: EDWARDS LIFESCIENCES CORPORATION, Irvine, CA (US)

(72) Inventor: Michael G. Valdez, Riverside, CA (US)

(21) Appl. No.: 18/365,809

(22) Filed: Aug. 4, 2023

Related U.S. Application Data
(63) Continuation of application No. PCT/US22/14931, filed on Feb. 2, 2022.
(60) Provisional application No. 63/146,263, filed on Feb. 5, 2021.

(51) Int. Cl. A61B 5/00; A61B 5/07; A61B 5/0215

(52) U.S. Cl. CPC A61B 5/6882; A61B 5/076; A61B 5/6869; A61B 5/0215; A61B 2562/12; A61M 27/002

(57) ABSTRACT

A sensor implant device includes a shunt body that forms a fluid conduit, a first anchor structure associated with a first end of the shunt body, a second anchor structure associated with a second end of the shunt body, a sensor device coupled to the first anchor structure, and an antenna coupled to the second anchor structure.

### RELATED APPLICATIONS

This application is a continuation of International Patent Application No. PCT/US2022/014931, filed Feb. 2, 2022 and entitled SHUNT SENSOR IMPLANT DEVICES, which claims priority to U.S. Provisional Patent Application No. 63/146,263, filed Feb. 5, 2021 and entitled SHUNT SENSOR IMPLANT DEVICES, the complete disclosures of which are hereby incorporated herein by reference in their entirety.

### BACKGROUND

### Field

The present disclosure generally relates to the field of medical implant devices.

### Description of Related Art

Various medical procedures involve the implantation of medical implant devices within the anatomy of the heart. Certain physiological parameters associated with such anatomy, such as fluid pressure, can have an impact on patient health prospects.

### SUMMARY

Described herein are one or more methods and/or devices to facilitate monitoring of physiological parameter(s) associated with certain chambers and/or vessels of the heart, such as the left atrium, using one or more sensor implant devices.

In some implementations, the present disclosure relates to a sensor implant device comprising a shunt body that forms a fluid conduit, a first anchor structure associated with a first end of the shunt body, a second anchor structure associated with a second end of the shunt body, a sensor device coupled to the first anchor structure, and an antenna coupled to the second anchor structure.

The sensor implant device may further comprise an electrical connector electrically connecting the sensor device to the antenna. For example, the electrical connector may be disposed at least partially within the fluid conduit of the shunt body. In some embodiments, the antenna comprises a coil wrapped around a magnetic core.

In some embodiments, the first anchor structure comprises one or more anchor arms configured to extend radially outward with respect to an axis of the fluid conduit and the sensor device is coupled to one of the one or more anchor arms. For example, the sensor device can include a sensor transducer including a sensor membrane that faces in a direction within 30° of the axis of the fluid conduit.

The shunt body can comprise a frame having a plurality of apertures therein. In some embodiments, the first anchor structure and the second anchor structure are configured to hold a portion of a tissue wall therebetween and, when the first anchor structure and the second anchor structure are holding the portion of the tissue wall, the portion of the tissue wall is disposed between the sensor device and the antenna.

The sensor implant device of claim 1, wherein, when the first anchor structure and the second anchor structure are projected radially outward with respect to an axis of the fluid conduit, the sensor device and the antenna are radially outside of an axial channel of the fluid conduit. For example, in some embodiments, when the first anchor structure and the second anchor structure are projected axially with respect to the axis of the fluid conduit in a delivery configuration of the sensor implant device, the sensor device and the antenna are within the axial channel of the fluid conduit.

In some implementations, the present disclosure relates to a sensor assembly comprising a sensor device configured to be attached to a first anchor of a prosthetic shunt implant device, an antenna coil configured to be attached to a second anchor of the prosthetic implant device, and an electrical connector coupled between the sensor device and the antenna coil.

The electrical connector may be dimensioned to extend through a tissue wall separating the sensor device and the antenna coil. In some embodiments, the antenna coil is configured to receive sensor signals from the sensor device over the electrical connector and transmit the sensor signals wirelessly.

In some implementations, the present disclosure relates to a sensor implant device comprising a tubular frame, a first anchor means associated with a first end of the tubular frame, a second anchor means associated with a second end of the tubular frame, a sensor device coupled to the first anchor means, and a wireless transmitter means coupled to the second anchor means.

The sensor implant device may further comprise a wire electrically connecting the sensor device to the transmitter means. For example, the wire may axially traverse the tubular frame. In some embodiments, the wire runs inside the tubular frame between the first end and the second end.

In some embodiments, the wireless transmitter means comprises a conductive coil. For example, the conductive coil may be wrapped around a cylindrical magnetic core.

In some embodiments, the first anchor means comprises a first anchor arm configured to extend radially outward with respect to an axis of the tubular frame, the sensor device is coupled to the first anchor arm, the second anchor means comprises a second anchor arm configured to extend radially outward with respect to the axis of the tubular frame, and the transmitter means in coupled to the second anchor arm.

In some embodiments, when the first anchor means and the second anchor means are projected radially outward with respect to an axis of the tubular frame, the sensor device and the antenna are radially outside of the tubular frame. For example, when the first anchor means and the second anchor means are oriented axially with respect to the axis of the tubular frame, the sensor device and the antenna may be radially within the tubular frame.

In some implementations, the present disclosure relates to a method of shunting fluid. The method comprises advancing a shunt implant device to a tissue wall within a delivery catheter, forming an opening in the tissue wall, deploying a first anchor structure of the shunt implant device on a distal side of the tissue wall, the first anchor structure having coupled thereto a sensor device, deploying a body of the shunt implant device in the opening in the tissue wall, and deploying a second anchor structure of the shunt implant device on a proximal side of the tissue wall, the second anchor structure having coupled thereto an antenna.

In some embodiments, the distal side of the tissue wall is within a left atrium of a heart and the proximal side of the tissue wall is within a coronary sinus of the heart.

In some implementations, the present disclosure relates to a method of manufacturing a shunt implant device. The method comprises forming a shunt structure including a shunt body configured to form a tubular conduit, a first anchor structure associated with a first axial end of the shunt body, and a second anchor structure associated with a second axial end of the shunt body, coupling a sensor device to the first anchor structure, and coupling an antenna to the second anchor structure.

The sensor device may be tethered to the antenna by an electrical connector. For example, the method may further comprise running the electrical connector through an interior of the tubular conduit.

For purposes of summarizing the disclosure, certain aspects, advantages, and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example representation of a human heart in accordance with one or more embodiments.

FIG. 2 illustrates example pressure waveforms associated with various chambers and vessels of the heart according to one or more embodiments.

FIG. 3 illustrates a graph showing left atrial pressure ranges.

FIG. 4 is a block diagram representing an implant device in accordance with one or more embodiments.

FIG. 5 is a block diagram representing a system for monitoring one or more physiological parameters associated with a patient according to one or more embodiments.

FIG. 6 illustrates an example shunt structure in accordance with one or more embodiments.

FIG. 7 shows a shunt structure implanted in an atrial septum in accordance with one or more embodiments.

FIG. 8 shows a sensor implant device implanted in a tissue wall between a coronary sinus and a left atrium in accordance with one or more embodiments.

FIG. 9-1 illustrates a side view of a sensor implant device in accordance with one or more embodiments.

FIG. 9-2 illustrates a sensor assembly in accordance with one or more embodiments.

FIG. 10 illustrates an axial views an embodiment of a shunt-type medical implant device having a sensor device secured at least in part thereto in accordance with one or more embodiments.

FIG. 11 illustrates an axial views an embodiment of a shunt-type medical implant device having a sensor device secured at least in part thereto in accordance with one or more embodiments.

FIG. 12 illustrates a sensor implant device implanted in a coronary sinus tissue wall with a sensor thereof exposed in a left atrium in accordance with one or more embodiments.

FIG. 13 illustrates a sensor implant device implanted in a coronary sinus tissue wall with a sensor thereof exposed in the coronary sinus in accordance with one or more embodiments.

FIG. 14 shows a sensor implant device implanted in an atrial septum with a sensor of the device exposed in a left atrium in accordance with one or more embodiments.

FIG. 15 shows a sensor implant device implanted in an atrial septum with a sensor of the device exposed in a right atrium in accordance with one or more embodiments.

FIGS. 16-1, 16-2, 16-3, 16-4, and 16-5 provide a flow diagram illustrating a process for implanting a sensor implant device in accordance with one or more embodiments.

FIGS. 17-1, 17-2, 17-3, 17-4, and 17-5 provide images of cardiac anatomy and certain devices/systems corresponding to operations of the process of FIGS. 16-1, 16-2, 16-3, 16-4, and 16-5 in accordance with one or more embodiments.

FIG. 18 is a cutaway view of a human heart and associated vasculature showing certain catheter access paths for pulmonary vein shunting procedures in accordance with one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for monitoring of one or more physiological parameters of a patient (e.g., blood pressure) using sensor-integrated cardiac shunts and/or other medical implant devices. In some implementations, the present disclosure relates to cardiac shunts and/or other cardiac implant devices that incorporate or are associated with pressure sensors or other sensor devices. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. Certain embodiments are disclosed herein in the context of cardiac implant devices. However, although certain principles disclosed herein are particularly applicable to the anatomy of the heart, it should be understood that sensor implant devices in accordance with the present disclosure may be implanted in, or configured for implantation in, any suitable or desirable anatomy.

### Cardiac Physiology

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

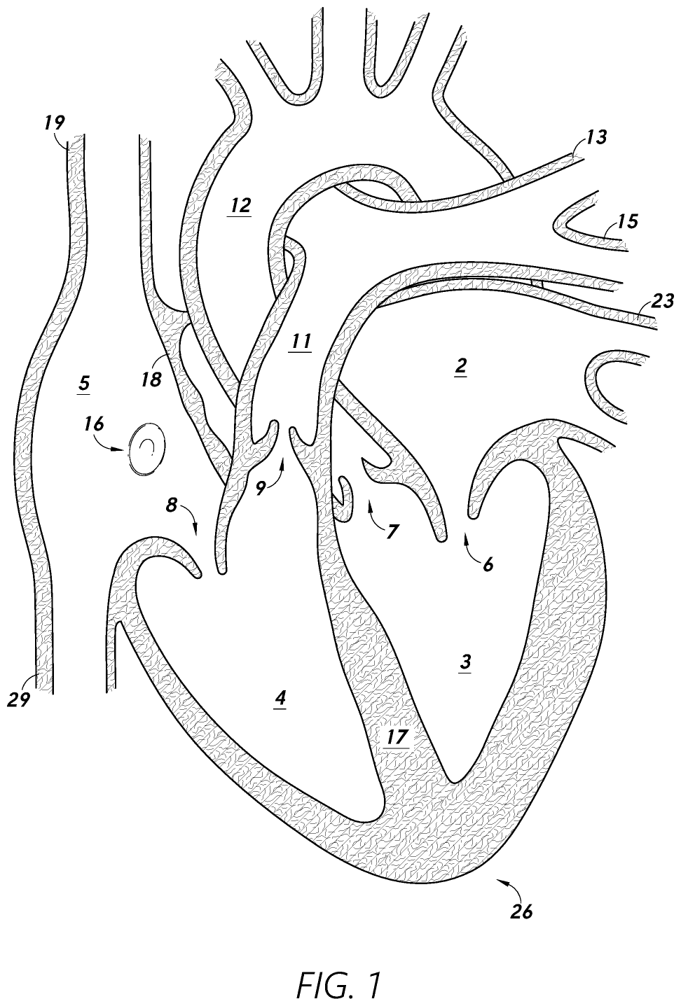

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle, referred to as the septum, separates the left-side chambers from the right-side chambers. In particular, an atrial septum wall portion 18 (referred to herein as the “atrial septum,” “interatrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 14 of the heart 1 is referred to as the apex and is generally located on or near the midclavicular line, in the fifth intercostal space.

The coronary sinus 16 comprises a collection of veins joined together to form a large vessel that collects blood from the heart muscle (myocardium). The ostium of the coronary sinus, which can be guarded at least in part by a Thebesian valve in some patients, is open to the right atrium 5, as shown. The coronary sinus runs along a posterior aspect of the left atrium 2 and delivers less-oxygenated blood to the right atrium 5. The coronary sinus generally runs transversely in the left atrioventricular groove on the posterior side of the heart.

Health Conditions Associated with Cardiac Pressure and Other Parameters

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

The treatment and/or prevention of heart failure (e.g., congestive heart failure) can advantageously involve the monitoring of pressure in one or more chambers or regions of the heart or other anatomy. As described above, pressure buildup in one or more chambers or areas of the heart can be associated with congestive heart failure. Without direct or indirect monitoring of cardiac pressure, it can be difficult to infer, determine, or predict the presence or occurrence of congestive heart failure. For example, treatments or approaches not involving direct or indirect pressure monitoring may involve measuring or observing other present physiological conditions of the patient, such as measuring body weight, thoracic impedance, right heart catheterization, or the like. In some solutions, pulmonary capillary wedge pressure can be measured as a surrogate of left atrial pressure. For example, a pressure sensor may be disposed or implanted in the pulmonary artery, and readings associated therewith may be used as a surrogate for left atrial pressure. However, with respect to catheter-based pressure measurement in the pulmonary artery or certain other chambers or regions of the heart, use of invasive catheters may be required to maintain such pressure sensors, which may be uncomfortable or difficult to implement. Furthermore, certain lung-related conditions may affect pressure readings in the pulmonary artery, such that the correlation between pulmonary artery pressure and left atrial pressure may be undesirably attenuated. As an alternative to pulmonary artery pressure measurement, pressure measurements in the right ventricle outflow tract may relate to left atrial pressure as well. However, the correlation between such pressure readings and left atrial pressure may not be sufficiently strong to be utilized in congestive heart failure diagnostics, prevention, and/or treatment.

Additional solutions may be implemented for deriving or inferring left atrial pressure. For example, the E/A ratio, which is a marker of the function of the left ventricle of the heart representing the ratio of peak velocity blood flow from gravity in early diastole (the E wave) to peak velocity flow in late diastole caused by atrial contraction (the A wave), can be used as a surrogate for measuring left atrial pressure. The E/A ratio may be determined using echocardiography or other imaging technology; generally, abnormalities in the E/A ratio may suggest that the left ventricle cannot fill with blood properly in the period between contractions, which may lead to symptoms of heart failure, as explained above. However, E/A ratio determination generally does not provide absolute pressure measurement values.

Various methods for identifying and/or treating congestive heart failure involve the observation of worsening congestive heart failure symptoms and/or changes in body weight. However, such signs may appear relatively late and/or be relatively unreliable. For example, daily bodyweight measurements may vary significantly (e.g., up to 9% or more) and may be unreliable in signaling heart-related complications. Furthermore, treatments guided by monitoring signs, symptoms, weight, and/or other biomarkers have not been shown to substantially improve clinical outcomes. In addition, for patients that have been discharged, such treatments may necessitate remote telemedicine systems.

The present disclosure provides systems, devices, and methods for guiding the administration of medication relating to the treatment of congestive heart failure at least in part by directly monitoring pressure in the left atrium, or other chamber or vessel for which pressure measurements are indicative of left atrial pressure and/or pressure levels in one or more other vessels/chambers, such as for congestive heart failure patients in order to reduce hospital readmissions, morbidity, and/or otherwise improve the health prospects of the patient.

### Cardiac Pressure Monitoring

Cardiac pressure monitoring in accordance with embodiments of the present disclosure may provide a proactive intervention mechanism for preventing or treating congestive heart failure and/or other physiological conditions. Generally, increases in ventricular filling pressures associated with diastolic and/or systolic heart failure can occur prior to the occurrence of symptoms that lead to hospitalization. For example, cardiac pressure indicators may present weeks prior to hospitalization with respect to some patients. Therefore, pressure monitoring systems in accordance with embodiments of the present disclosure may advantageously be implemented to reduce instances of hospitalization by guiding the appropriate or desired titration and/or administration of medications before the onset of heart failure.

Dyspnea represents a cardiac pressure indicator characterized by shortness of breath or the feeling that one cannot breathe well enough. Dyspnea may result from elevated atrial pressure, which may cause fluid buildup in the lungs from pressure back-up. Pathological dyspnea can result from congestive heart failure. However, a significant amount of time may elapse between the time of initial pressure elevation and the onset of dyspnea, and therefore symptoms of dyspnea may not provide sufficiently-early signaling of elevated atrial pressure. By monitoring pressure directly according to embodiments of the present disclosure, normal ventricular filling pressures may advantageously be maintained, thereby preventing or reducing effects of heart failure, such as dyspnea.

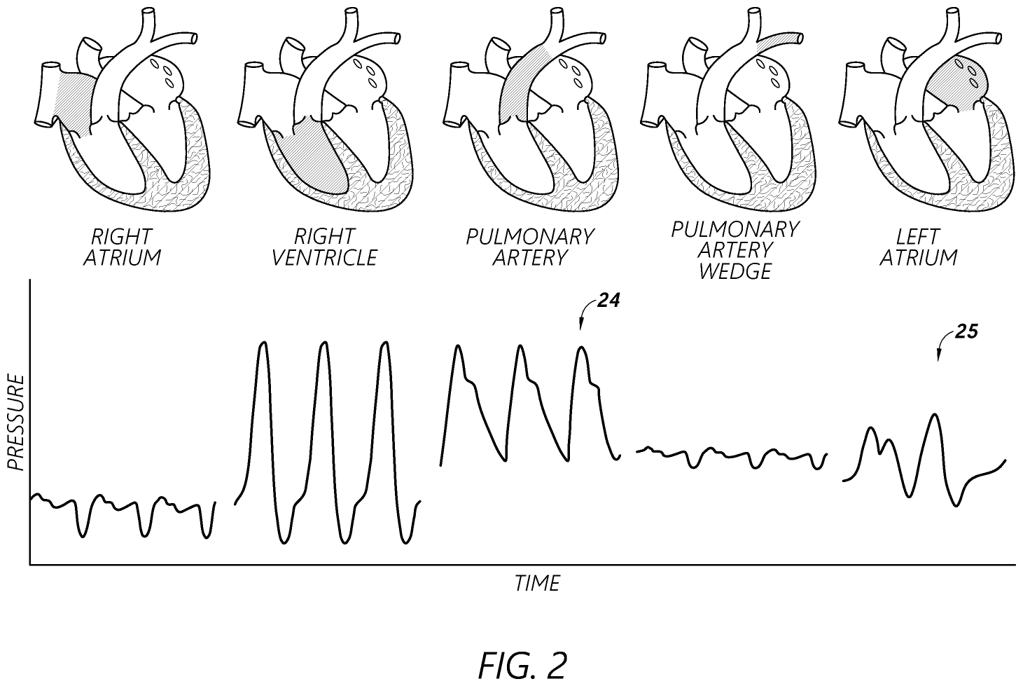

As referenced above, with respect to cardiac pressures, pressure elevation in the left atrium may be particularly correlated with heart failure. FIG. 2 illustrates example pressure waveforms associated with various chambers and vessels of the heart according to one or more embodiments. The various waveforms illustrated in FIG. 2 may represent waveforms obtained using right heart catheterization to advance one or more pressure sensors to the respective illustrated and labeled chambers or vessels of the heart. As illustrated in FIG. 2, the waveform 25, which represents left atrial pressure, may be considered to provide the best feedback for early detection of congestive heart failure. Furthermore, there may generally be a relatively strong correlation between increases and left atrial pressure and pulmonary congestion.

Left atrial pressure may generally correlate well with left ventricular end-diastolic pressure. However, although left atrial pressure and end-diastolic pulmonary artery pressure can have a significant correlation, such correlation may be weakened when the pulmonary vascular resistance becomes elevated. That is, pulmonary artery pressure generally fails to correlate adequately with left ventricular end-diastolic pressure in the presence of a variety of acute conditions, which may include certain patients with congestive heart failure. For example, pulmonary hypertension, which affects approximately 25% to 83% of patients with heart failure, can affect the reliability of pulmonary artery pressure measurement for estimating left-sided filling pressure. Therefore, pulmonary artery pressure measurement alone, as represented by the waveform 24, may be an insufficient or inaccurate indicator of left ventricular end-diastolic pressure, particularly for patients with co-morbidities, such as lung disease and/or thromboembolism. Left atrial pressure may further be correlated at least partially with the presence and/or degree of mitral regurgitation.

Left atrial pressure readings may be relatively less likely to be distorted or affected by other conditions, such as respiratory conditions or the like, compared to the other pressure waveforms shown in FIG. 2. Generally, left atrial pressure may be significantly predictive of heart failure, such as up two weeks before manifestation of heart failure. For example, increases in left atrial pressure, and both diastolic and systolic heart failure, may occur weeks prior to hospitalization, and therefore knowledge of such increases may be used to predict the onset of congestive heart failure, such as acute debilitating symptoms of congestive heart failure.

Cardiac pressure monitoring, such as left atrial pressure monitoring, can provide a mechanism to guide administration of medication to treat and/or prevent congestive heart failure. Such treatments may advantageously reduce hospital readmissions and morbidity, as well as provide other benefits. An implanted pressure sensor in accordance with embodiments of the present disclosure may be used to predict heart failure up two weeks or more before the manifestation of symptoms or markers of heart failure (e.g., dyspnea). When heart failure predictors are recognized using cardiac pressure sensor embodiments in accordance with the present disclosure, certain prophylactic measures may be implemented, including medication intervention, such as modification to a patient's medication regimen, which may help prevent or reduce the effects of cardiac dysfunction. Direct pressure measurement in the left atrium can advantageously provide an accurate indicator of pressure buildup that may lead to heart failure or other complications. For example, trends of atrial pressure elevation may be analyzed or used to determine or predict the onset of cardiac dysfunction, wherein drug or other therapy may be augmented to cause reduction in pressure and prevent or reduce further complications.

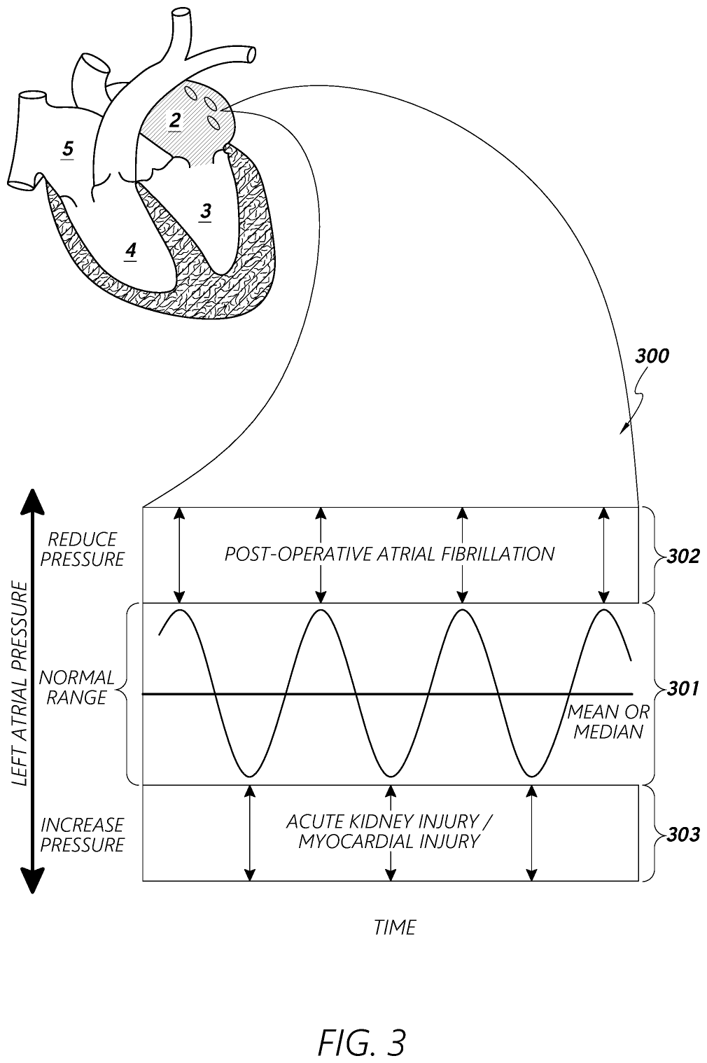

FIG. 3 illustrates a graph 300 showing left atrial pressure ranges including a normal range 301 of left atrial pressure that is not generally associated with substantial risk of postoperative atrial fibrillation, acute kidney injury, myocardial injury, heart failure and/or other health conditions. Embodiments of the present disclosure provide systems, devices, and methods for determining whether a patient's left atrial pressure is within the normal range 301, above the normal range 303, or below the normal range 302 through the use of certain sensor implant devices. For detected left atrial pressure above the normal range, which may be correlated with an increased risk of heart failure, embodiments of the present disclosure as described in detail below can inform efforts to reduce the left atrial pressure until it is brought within the normal range 301. Furthermore, for detected left atrial pressure that is below the normal range 301, which may be correlated with increased risks of acute kidney injury, myocardial injury, and/or other health complications, embodiments of the present disclosure as described in detail below can serve to facilitate efforts to increase the left atrial pressure to bring the pressure level within the normal range 301.

Implant Devices with Integrated Sensors

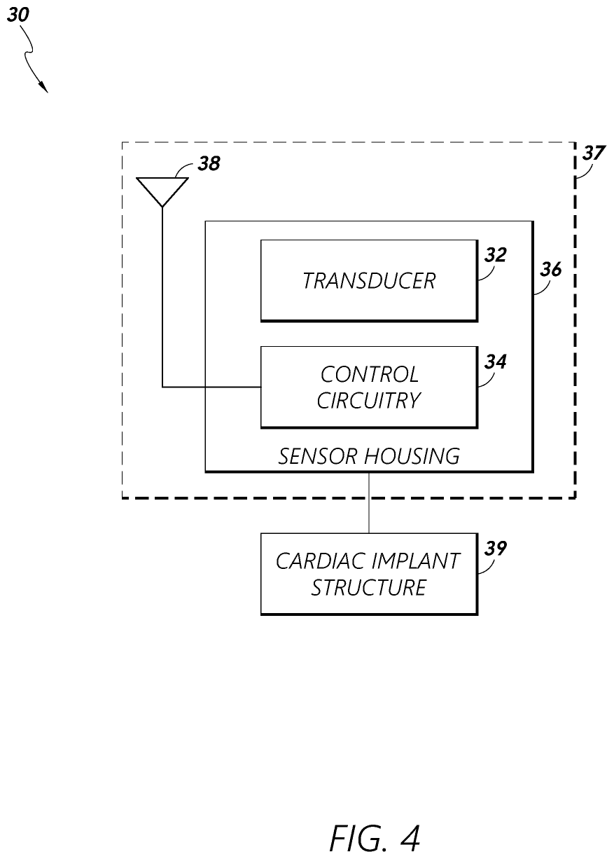

In some implementations, the present disclosure relates to sensors associated or integrated with cardiac shunts or other implant devices. Such integrated devices may be used to provide controlled and/or more effective therapies for treating and preventing heart failure and/or other health complications related to cardiac function. FIG. 4 is a block diagram illustrating an implant device 30 comprising a shunt (or other type of implant) structure 39. In some embodiments, the shunt structure 39 is physically integrated with and/or connected to a sensor device 37. The sensor device 37 may be, for example, a pressure sensor, or other type of sensor. In some embodiments, the sensor 37 comprises a transducer 32, such as a pressure transducer, as well as certain control circuitry 34, which may be embodied in, for example, an application-specific integrated circuit (ASIC).

The control circuitry 34 may be configured to process signals received from the transducer 32 and/or communicate signals associated therewith wirelessly through biological tissue using the antenna 38. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry. The transducer(s) 32 and/or antenna(s) 38 can be considered part of the control circuitry 34.

The antenna 38 may comprise one or more coils or loops of conductive material, such as copper wire or the like. In some embodiments, at least a portion of the transducer 32, control circuitry 34, and/or the antenna 38 are at least partially disposed or contained within a sensor housing 36, which may comprise any type of material, and may advantageously be at least partially hermetically sealed. For example, the housing 36 may comprise glass or other rigid material in some embodiments, which may provide mechanical stability and/or protection for the components housed therein. In some embodiments, the housing 36 is at least partially flexible. For example, the housing may comprise polymer or other flexible structure/material, which may advantageously allow for folding, bending, or collapsing of the sensor 37 to allow for transportation thereof through a catheter or other introducing means.

The transducer 32 may comprise any type of sensor means or mechanism. For example, the transducer 32 may be a force-collector-type pressure sensor. In some embodiments, the transducer 32 comprises a diaphragm, piston, bourdon tube, bellows, or other strain- or deflection-measuring component(s) to measure strain or deflection applied over an area/surface thereof. The transducer 32 may be associated with the housing 36, such that at least a portion thereof is contained within or attached to the housing 36. With respect to sensor devices/components being “associated with” a stent or other implant structure, such terminology may refer to a sensor device or component being physically coupled, attached, or connected to, or integrated with, the implant structure.

In some embodiments, the transducer 32 comprises or is a component of a piezoresistive strain gauge, which may be configured to use a bonded or formed strain gauge to detect strain due to applied pressure, wherein resistance increases as pressure deforms the component/material. The transducer 32 may incorporate any type of material, including but not limited to silicon (e.g., monocrystalline), polysilicon thin film, bonded metal foil, thick film, silicon-on-sapphire, sputtered thin film, and/or the like.

In some embodiments, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicon, and the like. In some embodiments, the transducer 32 comprises or is a component of an electromagnetic pressure sensor, which may be configured to measure the displacement of a diaphragm by means of changes in inductance, linear variable displacement transducer (LVDT) functionality, Hall Effect, or eddy current sensing. In some embodiments, the transducer 32 comprises or is a component of a piezoelectric strain sensor. For example, such a sensor may determine strain (e.g., pressure) on a sensing mechanism based on the piezoelectric effect in certain materials, such as quartz.

In some embodiments, the transducer 32 comprises or is a component of a strain gauge. For example, a strain gauge embodiment may comprise a pressure sensitive element on or associated with an exposed surface of the transducer 32. In some embodiments, a metal strain gauge is adhered to a surface of the sensor, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

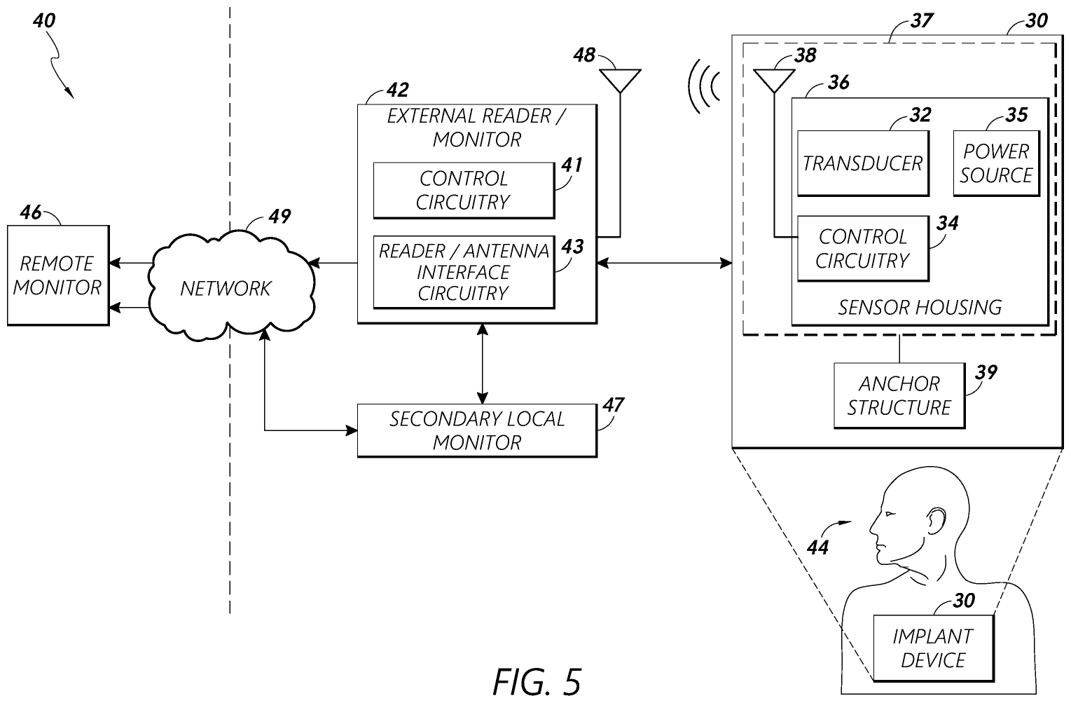

FIG. 5 shows a system 40 for monitoring one or more physiological parameters (e.g., left atrial pressure and/or volume) in a patient 44 according to one or more embodiments. The patient 44 can have a medical implant device 30 implanted in, for example, the heart (not shown), or associated physiology, of the patient 44. For example, the implant device 30 can be implanted at least partially within the left atrium and/or coronary sinus of the patient's heart. The implant device 30 can include one or more sensor transducers 32, such as one or more microelectromechanical system (MEMS) devices (e.g., MEMS pressure sensors, or other type of sensor transducer).

In certain embodiments, the monitoring system 40 can comprise at least two subsystems, including an implantable internal subsystem or device 30 that includes the sensor transducer(s) 32, as well as control circuitry 34 comprising one or more microcontroller(s), discrete electronic component(s), and one or more power and/or data transmitter(s) 38 (e.g., antennae coil). The monitoring system 40 can further include an external (e.g., non-implantable) subsystem that includes an external reader 42 (e.g., coil), which may include a wireless transceiver that is electrically and/or communicatively coupled to certain control circuitry 41. In certain embodiments, both the internal 30 and external 42 subsystems include a corresponding coil antenna for wireless communication and/or power delivery through patient tissue disposed therebetween. The sensor implant device 30 can be any type of implant device. For example, in some embodiments, the implant device 30 comprises a pressure sensor integrated with another functional implant structure 39, such as a prosthetic shunt or stent device/structure.

Certain details of the implant device 30 are illustrated in the enlarged block 30 shown. The implant device 30 can comprise an implant/anchor structure 39 as described herein. For example, the implant/anchor structure 39 can include a percutaneously-deliverable shunt device configured to be secured to and/or in a tissue wall to provide a flow path between two chambers and/or vessels of the heart, as described in detail throughout the present disclosure. Although certain components are illustrated in FIG. 5 as part of the implant device 30, it should be understood that the sensor implant device 30 may only comprise a subset of the illustrated components/modules and can comprise additional components/modules not illustrated. The implant device may represent an embodiment of the implant device shown in FIG. 4, and vice versa. The implant device 30 can advantageously include one or more sensor transducers 32, which can be configured to provide a response indicative of one or more physiological parameters of the patient 44, such as atrial pressure. Although pressure transducers are described, the sensor transducer(s) 32 can comprise any suitable or desirable types of sensor transducer(s) for providing signals relating to physiological parameters or conditions associated with the implant device 30 and/or patient 44.

The sensor transducer(s) 32 can comprise one or more MEMS sensors, optical sensors, piezoelectric sensors, electromagnetic sensors, strain sensors/gauges, accelerometers, gyroscopes, diaphragm-based sensors, and/or other types of sensors, which can be positioned in the patient 44 to sense one or more parameters relevant to the health of the patient. The transducer 32 may be a force-collector-type pressure sensor. In some embodiments, the transducer 32 comprises a diaphragm, piston, bourdon tube, bellows, or other strain- or deflection-measuring component(s) to measure strain or deflection applied over an area/surface thereof. The transducer 32 may be associated with the sensor housing 36, such that at least a portion thereof is contained within, or attached to, the housing 36.

In some embodiments, the transducer 32 comprises or is a component of a strain gauge, which may be configured to use a bonded or formed strain gauge to detect strain due to applied pressure. For example, the transducer 32 may comprise or be a component of a piezoresistive strain gauge, wherein resistance increases as pressure deforms the component/material of the strain gauge. The transducer 32 may incorporate any type of material, including but not limited to silicone, polymer, silicon (e.g., monocrystalline), polysilicon thin film, bonded metal foil, thick film, silicon-on-sapphire, sputtered thin film, and/or the like. In some embodiments, a metal strain gauge is adhered to the sensor surface, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

In some embodiments, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicone, silicon or other semiconductor, and the like. In some embodiments, the transducer 32 comprises or is a component of an electromagnetic pressure sensor, which may be configured to measures the displacement of a diaphragm by means of changes in inductance, linear variable displacement transducer (LVDT) functionality, Hall Effect, or eddy current sensing. In some embodiments, the transducer 32 comprises or is a component of a piezoelectric strain sensor. For example, such a sensor may determine strain (e.g., pressure) on a sensing mechanism based on the piezoelectric effect in certain materials, such as quartz.

In some embodiments, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

In certain embodiments, the sensor transducer(s) 32 can be configured to generate electrical signals that can be wirelessly transmitted to a device outside the patient's body, such as the illustrated local external monitor system 42. In order to perform such wireless data transmission, the implant device 30 can include radio frequency (RF) (or other frequency band) transmission circuitry, such as signal processing circuitry and an antenna 38. The antenna 38 can comprise an antenna coil implanted within the patient. The control circuitry 34 may comprise any type of transceiver circuitry configured to transmit an electromagnetic signal, wherein the signal can be radiated by the antenna 38, which may comprise one or more conductive wires, coils, plates, or the like. The control circuitry 34 of the implant device 30 can comprise, for example, one or more chips or dies configured to perform some amount of processing on signals generated and/or transmitted using the device 30. However, due to size, cost, and/or other constraints, the implant device 30 may not include independent processing capability in some embodiments.

The wireless signals generated by the implant device 30 can be received by the local external monitor device or subsystem 42, which can include a reader/antenna-interface circuitry module 43 configured to receive the wireless signal transmissions from the implant device 30, which is disposed at least partially within the patient 44. For example, the module 43 may include transceiver device(s)/circuitry.

The external local monitor 42 can receive the wireless signal transmissions from the implant device 30 and/or provide wireless power to the implant device 30 using an external antenna 48, such as a wand device. The reader/antenna-interface circuitry 43 can include radio-frequency (RF) (or other frequency band) front-end circuitry configured to receive and amplify the signals from the implant device 30, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The reader/antenna-interface circuitry 43 can further be configured to transmit signals over a network 49 to a remote monitor subsystem or device 46. The RF circuitry of the reader/antenna-interface circuitry 43 can further include one or more of digital-to-analog converter (DAC) circuitry, power amplifiers, low-pass filters, antenna switch modules, antennas or the like for treatment/processing of transmitted signals over the network 49 and/or for receiving signals from the implant device 30. In certain embodiments, the local monitor 42 includes control circuitry 41 for performing processing of the signals received from the implant device 30. The local monitor 42 can be configured to communicate with the network 49 according to a known network protocol, such as Ethernet, Wi-Fi, or the like. In certain embodiments, the local monitor 42 comprises a smartphone, laptop computer, or other mobile computing device, or any other type of computing device.

In certain embodiments, the implant device 30 includes some amount of volatile and/or non-volatile data storage. For example, such data storage can comprise solid-state memory utilizing an array of floating-gate transistors, or the like. The control circuitry 34 may utilize data storage for storing sensed data collected over a period of time, wherein the stored data can be transmitted periodically to the local monitor 42 or another external subsystem. In certain embodiments, the implant device 30 does not include any data storage. The control circuitry 34 may be configured to facilitate wireless transmission of data generated by the sensor transducer(s) 32, or other data associated therewith. The control circuitry 34 may further be configured to receive input from one or more external subsystems, such as from the local monitor 42, or from a remote monitor 46 over, for example, the network 49. For example, the implant device 30 may be configured to receive signals that at least partially control the operation of the implant device 30, such as by activating/deactivating one or more components or sensors, or otherwise affecting operation or performance of the implant device 30.

The one or more components of the implant device 30 can be powered by one or more power sources 35. Due to size, cost and/or electrical complexity concerns, it may be desirable for the power source 35 to be relatively minimalistic in nature. For example, high-power driving voltages and/or currents in the implant device 30 may adversely affect or interfere with operation of the heart or other body part associated with the implant device. In certain embodiments, the power source 35 is at least partially passive in nature, such that power can be received from an external source wirelessly by passive circuitry of the implant device 30, such as through the use of short-range, or near-field wireless power transmission, or other electromagnetic coupling mechanism. For example, the local monitor 42 may serve as an initiator that actively generates an RF field that can provide power to the implant device 30, thereby allowing the power circuitry of the implant device to take a relatively simple form factor. In certain embodiments, the power source 35 can be configured to harvest energy from environmental sources, such as fluid flow, motion, or the like. Additionally or alternatively, the power source 35 can comprise a battery, which can advantageously be configured to provide enough power as needed over the monitoring period (e.g., 3, 5, 10, 20, 30, 40, or 90 days, or other period of time).

In some embodiments, the local monitor device 42 can serve as an intermediate communication device between the implant device 30 and the remote monitor 46. The local monitor device 42 can be a dedicated external unit designed to communicate with the implant device 30. For example, the local monitor device 42 can be a wearable communication device, or other device that can be readily disposed in proximity to the patient 44 and implant device 30. The local monitor device 42 can be configured to continuously, periodically, or sporadically interrogate the implant device 30 in order to extract or request sensor-based information therefrom. In certain embodiments, the local monitor 42 comprises a user interface, wherein a user can utilize the interface to view sensor data, request sensor data, or otherwise interact with the local monitor system 42 and/or implant device 30.

The system 40 can include a secondary local monitor 47, which can be, for example, a desktop computer or other computing device configured to provide a monitoring station or interface for viewing and/or interacting with the monitored cardiac pressure data. In an embodiment, the local monitor 42 can be a wearable device or other device or system configured to be disposed in close physical proximity to the patient and/or implant device 30, wherein the local monitor 42 is primarily designed to receive/transmit signals to and/or from the implant device 30 and provide such signals to the secondary local monitor 47 for viewing, processing, and/or manipulation thereof. The external local monitor system 42 can be configured to receive and/or process certain metadata from or associated with the implant device 30, such as device ID or the like, which can also be provided over the data coupling from the implant device 30.

The remote monitor subsystem 46 can be any type of computing device or collection of computing devices configured to receive, process and/or present monitor data received over the network 49 from the local monitor device 42, secondary local monitor 47, and/or implant device 30. For example, the remote monitor subsystem 46 can advantageously be operated and/or controlled by a healthcare entity, such as a hospital, doctor, or other care entity associated with the patient 44. Although certain embodiments disclosed herein describe communication with the remote monitor subsystem 46 from the implant device indirectly through the local monitor device 42, in certain embodiments, the implant device 30 can comprise a transmitter capable of communicating over the network 49 with the remote monitor subsystem 46 without the necessity of relaying information through the local monitor device 42.

In some embodiments, at least a portion of the transducer 32, control circuitry 34, power source 35 and/or the antenna 38 are at least partially disposed or contained within the sensor housing 36, which may comprise any type of material, and may advantageously be at least partially hermetically sealed. For example, the housing 36 may comprise glass or other rigid material in some embodiments, which may provide mechanical stability and/or protection for the components housed therein. In some embodiments, the housing 36 is at least partially flexible. For example, the housing may comprise polymer or other flexible structure/material, which may advantageously allow for folding, bending, or collapsing of the sensor 30 to allow for transportation thereof through a catheter or other percutaneous introducing means.

### Cardiac Shunt Implants

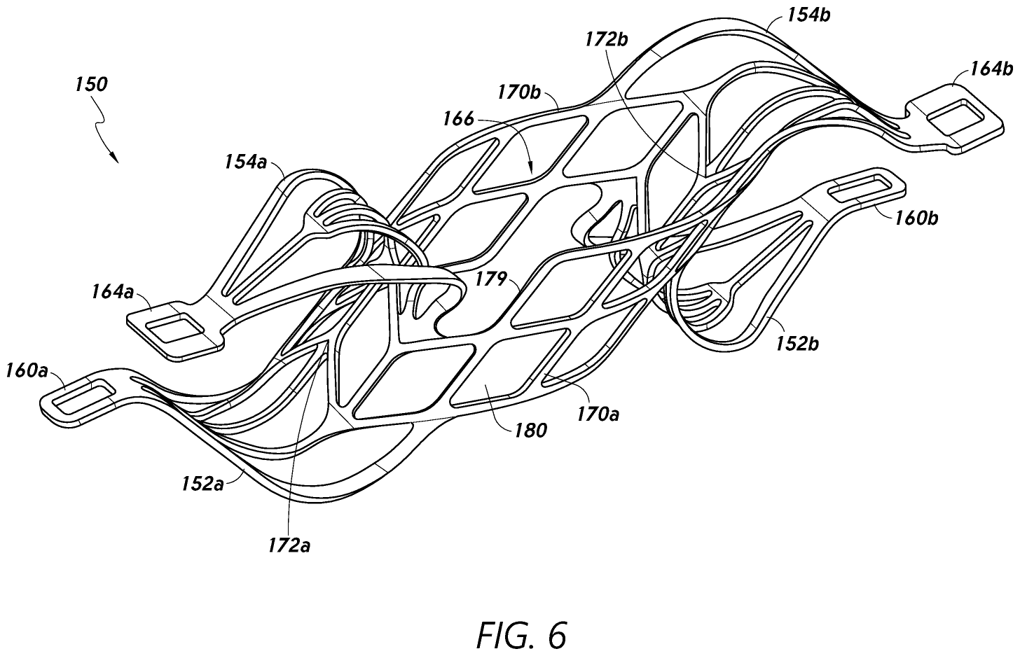

FIG. 6 illustrates an example shunt/anchor structure 150 in accordance with one or more embodiments. The shunt structure 150 may represent an embodiment of a cardiac implant (e.g., anchor and/or cardiac implant structure associated with FIG. 4 or 5) that may be integrated with pressure sensor functionality in accordance with certain embodiments disclosed herein. The shunt structure 150 may be an expandable shunt. When expanded, a central flow channel 166 of the shunt 150 may define a generally circular or oval opening. The channel 166 may be configured to hold the sides of a puncture opening in a tissue wall to form a blood flow path between chamber(s) or vessel(s) of the heart that are separated by the tissue wall. For example, the shunt 150 may be configured to be implanted in the wall separating the coronary sinus and the left atrium. The central flow channel 166 may be partly formed by a pair of side walls 170a, 170b defined by a generally parallel arrangement of thin struts 179 that forms an array of parallelogram-shaped cells or openings 180. In some embodiments, substantially the entire shunt 150 is formed by super-elastic struts that are configured to be compressed and fit into a catheter (not shown) and subsequently expanded back to the relaxed shape as shown in FIG. 6.

Formation of the shunt 150 using a plurality of interconnected struts forming cells therebetween may serve to at least partially increase the flexibility of the shunt, thereby enabling compression thereof and expansion at the implant site. The interconnected struts around the central flow channel 166 advantageously provide a cage having sufficient rigidity and structure to hold the tissue at the puncture in an open position. End walls 172a, 172b of the central flow channel 166 can serve to connect the side walls 170a, 170b and extend between distal and proximal flanges, or arms, 152, 154 on each side. The side walls 170a, 170b and end walls 172a, 172b together may define a tubular lattice, as shown. The end walls 172a, 172b can comprise thin struts 179 extending at a slight angle from a central flow axis of the shunt 150.

Although the illustrated shunt 150 comprises struts that define a tubular or circular lattice of open cells forming the central flow channel 166, in some embodiments, the structure that makes up the channel forms a substantially contiguous wall surface through at least a portion of the channel 166. In the illustrated embodiment, the tilt of the shunt structure 150 may facilitate collapse of the shunt into a delivery catheter (not shown), as well as the expansion of the flanges/arm 152, 154 on both sides of a target tissue wall. The central flow channel 166 may remain essentially unchanged between the collapsed and expanded states of the shunt 150, whereas the flanges/arms 152, 154 may transition in and out of alignment with the angled flow channel.

Although certain embodiments of shunts disclosed herein comprise flow channels having substantially circular cross-sections, in some embodiments, shunt structures in accordance with the present disclosure have oval-shaped, rectangular, diamond-shaped, or elliptical flow channel configuration. For example, relatively elongated side walls compared to the illustrated configuration of FIG. 5 may produce a rectangular or oval-shaped flow channel. Such shapes of shunt flow channels may be desirable for larger punctures, while still being configured to collapse down to a relatively small delivery profile.

In some embodiments, each of the distal and proximal flanges/arms 152, 154 is configured to curl outward from the end walls 172a, 172b and be set to point approximately radially away from the central flow channel 166 in the expanded configuration. The expanded flanges/arms may serve to secure the shunt 150 to a target tissue wall. Additional aspects and features of shunt, implant, and/or anchor structures that may be integrated with sensor devices/functionality of embodiments of the present disclosure are disclosed in U.S. Pat. No. 9,789,294, entitled “Expandable Cardiac Shunt,” issued on Oct. 17, 2017, the disclosure of which is hereby expressly incorporated by reference in its entirety. Although certain embodiments are disclosed herein in the context of shunt structures similar to that shown in FIG. 5 and described above, it should be understood that shunt structures or other implant devices integrated with pressure sensor functionality in accordance with embodiments of the present disclosure may have any type, form, structure, configuration, and/or may be used or configured to be used for any purpose, whether for shunting or other purpose or functionality.

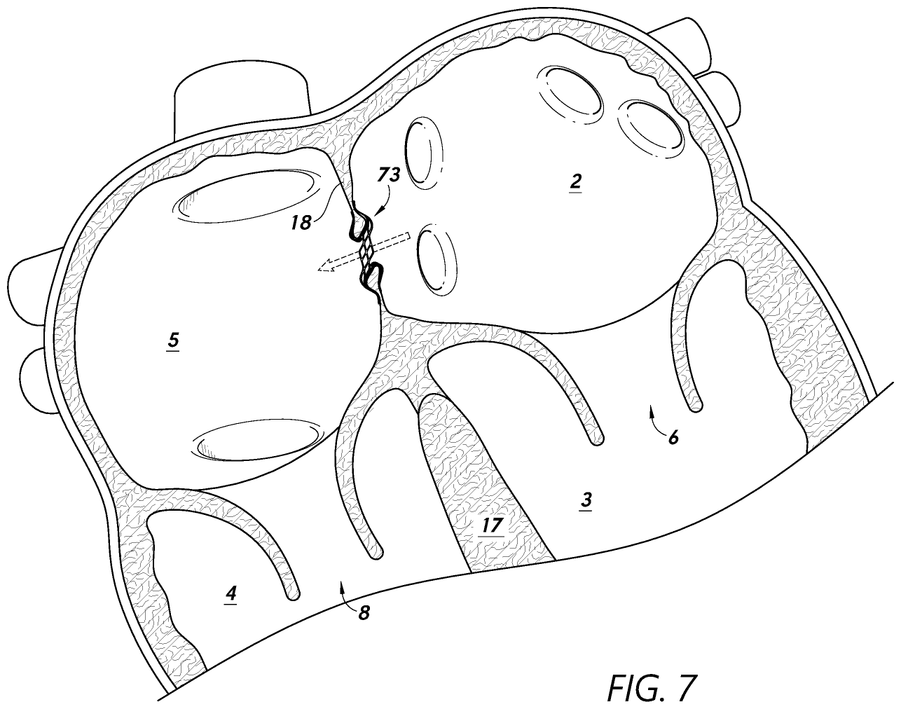

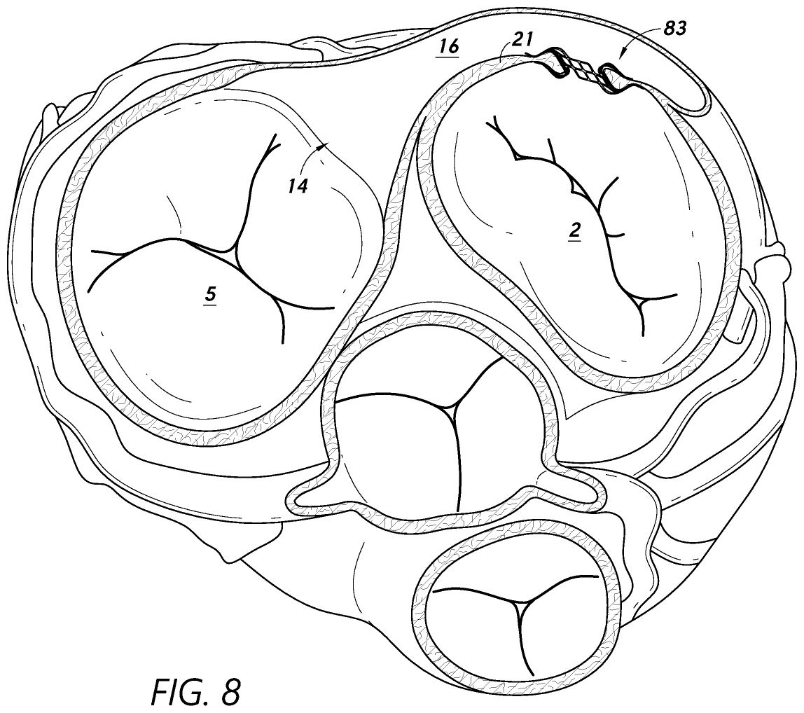

FIG. 7 shows a shunt implant/anchor device/structure 73 implanted in an atrial septum 18 in accordance with one or more embodiments. The particular position in the interatrial septum wall 18 may be selected or determined to provide a relatively secure anchor location for the shunt structure 73. Furthermore, the shunt device/structure 73 may be implanted at a position that is desirable in consideration of future re-crossing of the septal wall 18 for future interventions. Implantation of the shunt device/structure 73 in the interatrial septum wall 18 may advantageously allow for fluid communication between the left 2 and right 5 atria.

Interatrial shunting using the shunt device/structure 73 may be well-suited for patients that are relatively highly sensitive to atrial pressure increases. For example, as pressure increases in the ventricles and/or atria and is applied against the myocardial cells, the muscles of the heart may generally be prone to contract relatively harder to process the excess blood. Therefore, as the ventricle dilates or stretches, for patients with compromised contractility of the ventricle, such patients may become more sensitive to higher pressures in the ventricle and/or atria because the heart may be unable to adequately respond or react thereto. Furthermore, increases in left atrial pressure can results in dyspnea, and therefore reduction in left atrial pressure to reduce dyspnea and/or reduce incidences of hospital readmission may be desirable through interatrial shunting. For example, when the ventricle experiences dysfunction such that is unable to accommodate build-up in fluid pressure, such fluid may backup into the atria, thereby increasing atrial pressure. With respect to heart failure, minimization of left ventricular end-diastolic pressure may be paramount. Because left ventricular end-diastolic pressure can be related to left atrial pressure, backup of fluid in the atrium can cause backup of fluid in the lungs, thereby causing undesirable and/or dangerous fluid buildup in the lungs. Interatrial shunting, such as using shunt devices in accordance with embodiments of the present disclosure, can divert extra fluid in the left atrium to the right atrium, which may be able to accommodate the additional fluid due to the relatively high compliance in the right atrium.

In some implementations, shunt device/structure in accordance with embodiments of the present disclosure may be implanted in a wall separating the coronary sinus from the left atrium, such that interatrial shunting may be achieved through the coronary sinus. FIG. 8 shows a shunt device/structure 83 implanted in a tissue wall 21 between the coronary sinus 16 and the left atrium 2. FIG. 8, as well as a number of the following figures, shows a section of the heart from a top-down, superior perspective with the posterior aspect oriented at the top of the page.

In some cases, left-to-right shunting through implantation of the shunt device 83 in the wall 21 between the left atrium 2 and the coronary sinus 16 can be preferable to shunting through the interatrial septum. For example, shunting through the coronary sinus 16 can provide reduced risk of thrombus and embolism. The coronary sinus is less likely to have thrombus/emboli present for several reasons. First, the blood draining from the coronary vasculature into the right atrium 5 has just passed through capillaries, so it is essentially filtered blood. Second, the ostium 14 of the coronary sinus in the right atrium is often partially covered by a pseudo-valve called the Thebesian Valve (not shown). The Thebesian Valve is not always present, but some studies show it is present in most hearts and can block thrombus or other emboli from entering in the event of a spike in right atrium pressure. Third, the pressure gradient between the coronary sinus and the right atrium into which it drains is generally relatively low, such that thrombus or other emboli in the right atrium is likely to remain there. Fourth, in the event that thrombus/emboli do enter the coronary sinus, there will be a much greater gradient between the right atrium and the coronary vasculature than between the right atrium and the left atrium. Most likely, thrombus/emboli would travel further down the coronary vasculature until right atrium pressure returned to normal and then the emboli would return directly to the right atrium.

Some additional advantages to locating the shunt structure 83 between the left atrium and the coronary sinus is that this anatomy is generally more stable than the interatrial septal tissue. By diverting left atrial blood into the coronary sinus, sinus pressures may increase by a small amount. This would cause blood in the coronary vasculature to travel more slowly through the heart, increasing perfusion and oxygen transfer, which can be more efficient and also can help a dying heart muscle to recover. In addition, by implanting the shunt device/structure 83 in the wall of the coronary sinus 83, damage to the interatrial septum 18 may be prevented. Therefore, the interatrial septum 18 may be preserved for later transseptal access for alternate therapies. The preservation of transseptal access may be advantageous for various reasons. For example, heart failure patients often have a number of other comorbidities, such as atrial fibrillation and/or mitral regurgitation; certain therapies for treating these conditions require a transseptal access.

It should be noted, that in addition to the various benefits of placing the implant/structure 83 between the coronary sinus 16 and the left atrium 2, certain drawbacks may be considered. For example, by shunting blood from the left atrium 2 to the coronary sinus 16, oxygenated blood from the left atrium 2 may be passed to the right atrium 5 and/or non-oxygenated blood from the right atrium 5 may be passed to the left atrium 2, both of which may be undesirable with respect to proper functioning of the heart.

### Sensor-Integrated Implant Devices

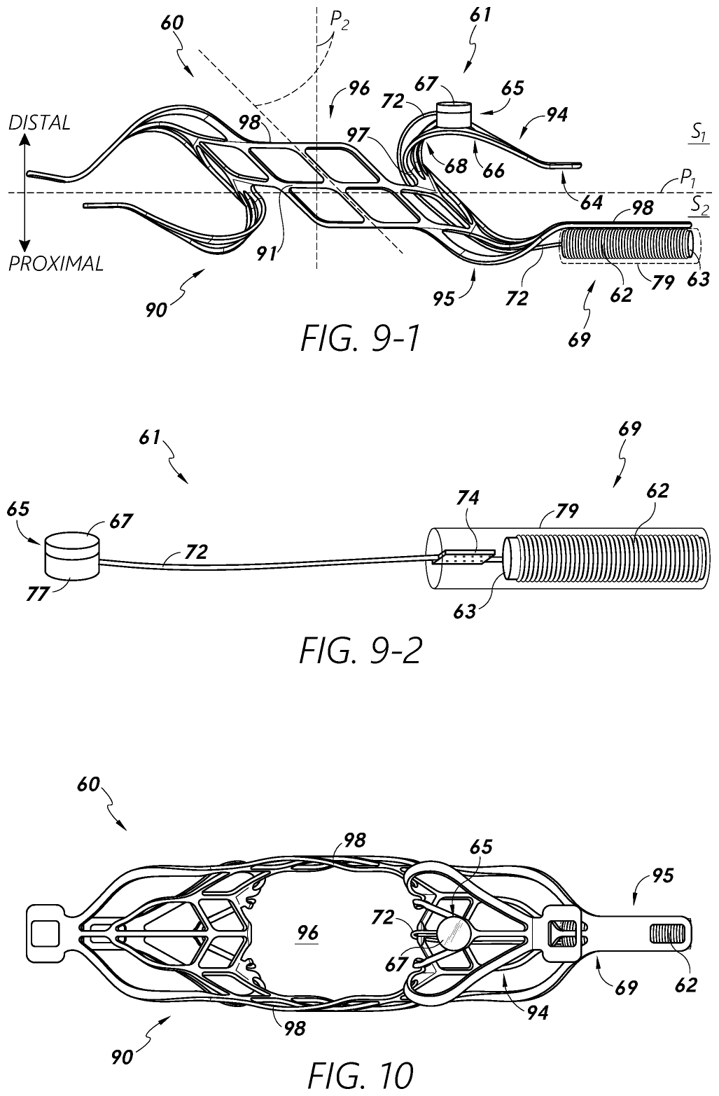

As referenced above, shunt and/or other implant devices/structures may be integrated with sensor, antenna/transceiver, and/or other components to facilitate in vivo monitoring of pressure and/or other physiological parameter(s). Sensor devices in accordance with embodiments of the present disclosure may be integrated with cardiac shunt structures/devices or other implant devices using any suitable or desirable attachment or integration mechanism or configuration. FIG. 9-1 illustrates a side view of a sensor implant device 60 comprising a shunt structure 90 and an integrated sensor assembly 61 in accordance with one or more embodiments. In some embodiments, the sensor assembly 61 may be built or manufactured into the shunt structure 90 to form a unitary structure, at least in part. FIG. 9-2 shows an isolated view of the sensor assembly 61.

In some embodiments, the sensor assembly 61 includes a sensor component 65 and an antenna component 69. The sensor component 65 may comprise any type of sensor device as described in detail above. In some embodiments, the sensor 65 may be attached to or integrated with an arm member 94 of the shunt structure 90, as shown. For example, the arm 94 with which the sensor component 65 is associated may be generally associated with a distal or proximal axial portion of the shunt structure 90. That is, when the shunt structure 90 is implanted, one or more arms of the shunt structure 90 may be associated with an inlet/distal portion of the shunt structure 90, whereas one or more other anchor arms (e.g., arm 95) may be associated with an outlet/proximal portion of the shunt structure 90. Although distal and proximal sides/portions are identified in FIG. 9-1, it should be understood that the identified distal portion/side may be an outlet or inlet side of the shunt structure 90, as with the identified proximal portion/side. Furthermore, the terms “distal” and “proximal” are used for convenience and may or may not refer to relative orientation with respect to a delivery system/device used to implant the sensor implant device 60 and/or shunt structure 90.

The sensor 65 includes a sensor element 67, such as a pressure sensor transducer. Relative to the arm member 94 of the shunt structure 90, the sensor device 65 may be attached/positioned at a distal 64, medial 66, or proximal 68 portion or area of the arm/anchor 94, or any portion therebetween. For example, the illustrated embodiment of FIG. 9-1 includes the sensor 65 disposed in the medial area 66 of the arm/anchor 94. In some embodiments, readings acquired by the sensor 65 may be used to guide titration of medication for treatment of a patient in whom the implant device 60 is implanted.

As described herein, the sensor assembly 61 may be configured to implement wireless data and/or power transmission. The sensor assembly 61 may include an antenna component 69 for such purpose. The antenna 69 may be contained at least partially within an antenna housing 79, which may further have disposed therein certain control circuitry configured to facilitate wireless data and/or power communication functionality. In some embodiments, the antenna component 69 comprises one or more conductive coils 62, which may facilitate inductive powering and/or data transmission. In embodiments comprising conductive coil(s), such coil(s) may be wrapped/disposed at least partially around a magnetic (e.g., ferrite, iron) core 63.

The antenna component 69 may be attached to, integrated with, or otherwise associated with an arm/anchor feature 95 of the shunt structure 90, the arm 95 being a separate arm/anchor from the arm/anchor 94 supporting the sensor device 65. For example, as with the sensor component 65, the antenna component 69 may be attached to or otherwise associated with one or more of a distal portion, medial portion, and/or proximal portion of the arm 95. In some embodiments, the arm 95 includes an elongated strut/arm feature 98 to which the antenna component 69 is secured, whereas the sensor device 65 may or may not be secured to a similar strut/arm feature of the arm 94 with which it is associated. For example, securement/attachment means/mechanisms that may be suitable for attaching the antenna component 69 and/or sensor component 65 to respective arms/anchors of the shunt structure 90 may be any of the features disclosed in PCT Application No. PCT/US20/56746, Filed on Oct. 22, 2020, and entitled “Sensor Integration in Cardiac Implant Devices,” the contents of which are hereby expressly incorporated by reference in their entirety. For example, the shunt structure 90 and/or arm(s) thereof may include one or more sensor and/or antenna retention fingers, clamps, wraps, bands, belts, clips, pouches, housings, encasements, and/or the like configured to secure the sensor component 65 and/or antenna component 69 to a respective arm and/or strut or other structural feature of the shunt structure 90.

As shown in FIG. 9-1, the sensor component 65 and antenna component 69 may advantageously be associated with opposite axial sides/ends/portions of the shunt structure 90, such that, when the shunt structure 90 is implanted in a tissue wall, the sensor device 65 and antenna device 69 may be disposed on opposite sides (S₁, S₂) of the tissue wall. Such a configuration may be advantageous for a variety of reasons. For example, by distributing the sensor device 65 and antenna device across opposite axial sides S₁/S₂ (e.g., distal/proximal and/or inlet/outlet sides) of the shunt structure 90, the structural bulkiness and/or profile of the sensor assembly 61 on any given side of the device 60 may be relatively less than it would be if the entire sensor assembly 61 was disposed on the same axial side of the device 60.

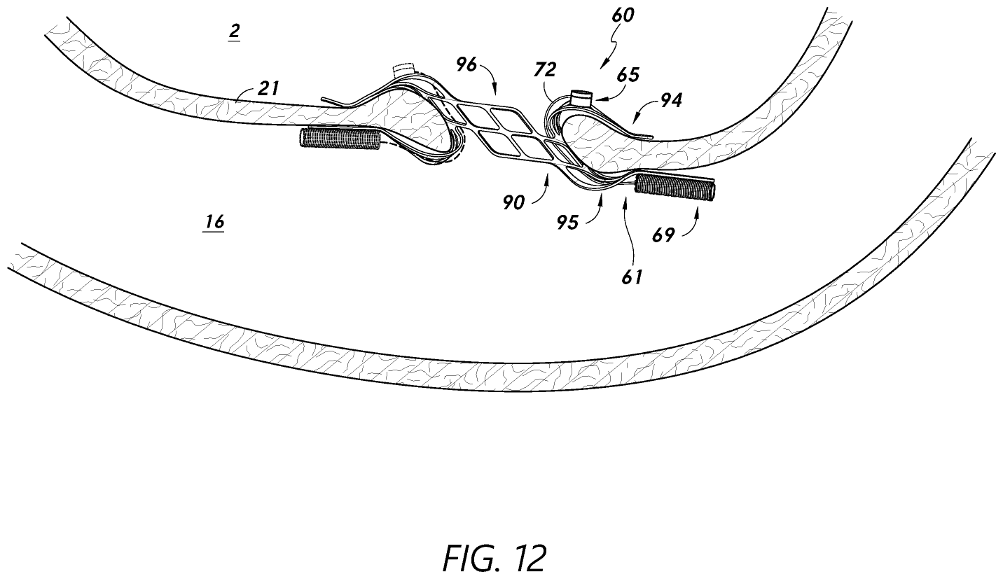

As described herein, references to axial sides of a shunt structure may refer to opposite sides of a plane P₁ axially (and/or diagonally, as in FIG. 9-1) bisecting the shunt structure 90 and/or barrel 98. The plane P₁ may be orthogonal to an axis of the barrel portion 98 of the shunt structure 90 and/or may be substantially parallel with a tissue wall in which the shunt structure 90 is implanted. That is, when the shunt structure 90 is implanted in a tissue wall (not shown in FIG. 9-1; see FIGS. 12-14), the axis of the barrel 98 may be askew/angled with respect to a line/plane that is normal to the tissue wall surface; it should be understood that description herein of shunt axes may be understood to refer to an axis/line that is substantially normal to a tissue wall engagement plane (e.g., plane P₁ shown in FIG. 9-1), even in embodiments/cases in which the shunt barrel has a true axis that is angled with respect to the tissue-engagement plane P₁ as in FIG. 9-1. Description herein of devices/components disposed on separate axial sides of an implant structure can be understood to refer to different sides of the tissue-engagement plane P₁. The plane P₁ may be aligned (e.g., within 5° or 10° of exact alignment) with at least some of the struts 91 of the barrel portion 98 of the shunt structure 90.

Furthermore, description herein of sensor assemblies and/or components thereof being disposed on different radial sides of a shunt structure may refer to diametrically opposite sides of a diametrical plane P₂, as shown in FIG. 9-1. For example, where a shunt structure includes arms on a given axial side of the shunt structure that emanate from substantially opposite circumferential portions of a barrel 98 of the shunt structure and/or project in substantially opposite radial directions with respect to an axis of the barrel 98 of the shunt structure, such arms may be considered to be on different and/or opposite radial sides of the shunt structure.

Disposing the sensor component 65 and antenna component 69 on opposite axial sides of the shunt structure 90 can further be preferable to allow for placement/exposure of the sensor transducer 67 in one chamber/vessel associated with one side S₁ of the tissue wall in which the implant device 60 is implanted, wherein the side S₁ is associated with a vessel/chamber that is a target for monitoring of pressure and/or other physiological parameter(s) relevant to the sensor component 65, whereas the antenna component 69 may be disposed in another vessel/chamber associated with the opposite side S₂ of the shunt structure 90 that is not necessarily the target/subject of monitoring. For example, in some instances, the fluid dynamics may be different in the target chamber/vessel on the sensor side S₁ than on the opposite side S₂ of the tissue wall in which the shunt structure 90 is implanted, wherein the fluid dynamics in the chamber/vessel on side S₂ associated with the antenna component 69 may be less turbulent than on side S₁. Furthermore, by placing the structure of the antenna component 69 on the opposite side S₂ from the sensor component 65, undesirable obstruction on the sensor target side S₁ may be avoided that might otherwise be introduced by placement of the antenna component 69 on the same axial side as the sensor component 65. Furthermore, placement of the sensor component 69 on the same axial side as the sensor component 65 may result in undesirable obstruction or interference with the sensor functionality and/or fluid dynamics associated with the fluid being sensed/monitored, such that the particular configuration shown in FIG. 9-1 may advantageously allow for integration of the sensor assembly 61 with the shunt structure 90 without undesirably interfering with or affecting the sensor functionality of the sensor device 65. Furthermore, by separating/distributing the sensor component 65 and the antenna component 69 as shown in FIG. 9-1 and described in detail herein, delivery of the device 60 in a delivery catheter/sheath and or other delivery system component(s) may be facilitated, and/or the sensor implant device 60 may be enabled to assume a relatively lower-profile delivery/compressed configuration, as described in greater detail below.

The sensor assembly 61 may further include an electrical connector 72, which may comprise one or more wires or other electrical conductors configured to transmit electrical signals between the sensor component 65 and the antenna component 69. In some embodiments, the connector 72 includes a coating or other covering configured to provide electrical and/or hermetic sealing/covering for the connector 72. In addition to providing electrical coupling between the sensor 65 in the antenna 69, the connector 72 may provide a physical tether/coupling between such components of the sensor assembly 61. The physical tethering/coupling of the sensor 65 and antenna 69 may provide a mechanism for protecting against the separation of either of the sensor 65 or antenna 69 from the sensor assembly 61, which could result in serious health complications that may result from the free movement of the component in the cardiac circulation. For example, if one of the sensor 65 or antenna 69 becomes dislodged or disconnected from the shunt structure 90 and/or sensor assembly 61, the connector 72 may prevent such disconnected component from becoming free within the circulation.

The connector 72 may have a length that is sufficient to allow for the distributed disposition of the sensor component 65 and antenna component 69 on opposite axial sides of the shunt structure, as shown. For example, the connector 72 may have a length of approximately 0.5″, or longer. For example, the connector 72 may have a length that is longer than necessary for the purpose of signal communication between the sensor component 65 antenna component 69 in order to provide a tether between such components having a length sufficient to span the axial length of the barrel 98 of the shunt structure 90, as well as certain proximal and/or medial portions of the respective arms 94, 95 of the shunt structure 90 separating the sensor 65 from the antenna 69.

Although FIG. 9-1 and various other depictions embodiments of the present disclosure relate to sensor assemblies in which a sensor component and antenna component are secured to the shunt/implant structure on opposite axial sides of the structure, in some embodiments, a sensor component and antenna component may be associated with the same axial side of a shunt/implant structure, wherein the sensor and antenna components are associated with opposite radial sides of the shunt/implant structure. For example, the connector electrically and/or physically coupling the sensor and anchor components may span the barrel 98 of the shunt structure 90, such as by crossing over the fluid channel 96 formed thereby, or may run along an outside or inside of the shunt barrel 98.

The sensor assembly 61 may advantageously be biocompatible. For example, the sensor 65 and antenna 69 may comprise biocompatible housings, such as a housing comprising glass or other biocompatible material. However, at least a portion of the sensor element 67, such as a diaphragm or other component, may be exposed to the external environment in some embodiments in order to allow for pressure readings, or other parameter sensing, to be implemented. With respect to the antenna housing 79, the housing 79 may comprise an at least partially rigid cylindrical or tube-like form, such as a glass cylinder form. In some embodiments, the sensor 65/67 component is approximately 3 mm or less in diameter. The antenna 69 may be approximately 20 mm or less in length.

The sensor assembly 61 may be configured to communicate with an external system when implanted in a heart or other area of a patient's body. For example, the antenna 69 may receive power wirelessly from the external system and/or communicate sensed data or waveforms to and/or from the external system. The sensor assembly 61 may be attached to, or integrated with, the shunt structure 90 in any suitable or desirable way. For example, in some implementations, the sensor 65 and/or antenna 69 may be attached or integrated with the shunt structure 90 using mechanical attachment means. In some embodiments, the sensor 65, connector 72, and/or antenna 69 may be contained in a pouch or other receptacle that is attached to the shunt structure 90.

The sensor element 67 may comprise a pressure transducer. For example, the pressure transducer may be a microelectromechanical system (MEMS) transducer comprising a semiconductor diaphragm component. In some embodiments, the transducer may include an at least partially flexible or compressible diaphragm component, which may be made from silicone or other flexible material. The diaphragm component may be configured to be flexed or compressed in response to changes in environmental pressure. The control circuitry 74 may be configured to process signals generated in response to said flexing/compression to provide pressure readings. In some embodiments, the diaphragm component is associated with a biocompatible layer on the outside surface thereof, such as silicon nitride (e.g., doped silicon nitride) or the like. The diaphragm component and/or other components of the pressure transducer 67 may advantageously be fused or otherwise sealed to/with a base/housing 77 of the sensor component 65 in order to provide hermetic sealing of at least some of the sensor assembly components.

The control circuitry 74 may comprise one or more electronic application-specific integrated circuit (ASIC) chips or die, which may be programmed and/or customized or configured to perform monitoring functionality as described herein and/or facilitate transmission of sensor signals wirelessly. The antenna 69 may comprise a ferrite core 63 wrapped with conductive material in the form of a plurality of coils 62 (e.g., wire coil). In some embodiments, the coils comprise copper or other metal. The antenna 69 may advantageously be configured with coil geometry that does not result in substantial displacement or heating in the presence of magnetic resonance imaging. In some implementations, the sensor implant device 60 may be delivered to a target implant site using a delivery catheter (not shown), wherein the delivery catheter includes a cavity or channel configured to accommodate the advancement of the sensor assembly 61 therethrough.

FIG. 10 illustrates an axial view of the implant device 60 of FIG. 9-1 in accordance with one or more embodiments of the present disclosure. Specifically, FIG. 10 shows an axial view that corresponds to an axial side of the implant device 60 associated with the sensor component 65. That is, the sensor component 65 is attached to, integrated with, or otherwise associated with the arm 94, the side of which is shown facing out of the page in FIG. 10. The side shown facing out of the page in FIG. 10 may be a distal or proximal side.

The sensor component 65 can be mechanically attached or fastened to a portion of the arm 94. The sensor 65 may be attached to the anchor/arm 94 by any suitable or desirable attachment means, including adhesive attachment or mechanical engagement. For example, the arm 94 may comprise or be associated with one or more retention features, which may comprise one or more clamps, straps, ties, sutures, collars, clips, tabs, or the like. Such retention features may circumferentially encase or retain the sensor 65, or a portion thereof. In some embodiments, the sensor 65 may be attached to the arm 94 through the application of mechanical force, either through sliding the sensor 65 through certain retention features or through clipping, locking, or otherwise engaging the sensor 65 with the arm 94 by pressing or applying other mechanical force thereto. In some embodiments, the shunt structure 90 may comprise one or more tabs that may be configured to pop-up or extend on one or more sides of the sensor 65 for mechanical fastening. Such tabs may comprise memory metal (e.g., Nitinol) or other at least partially rigid material. In some embodiments, the sensor 65 is pre-attached to the arm 94 and/or integrated therewith prior to implantation. In some embodiments, the sensor 65 may be built or manufactured into the shunt structure 90 to form a unitary structure. For example, in some embodiments, the sensor 65 may be attached to or integrated with the arm member 94 of the shunt structure 90.

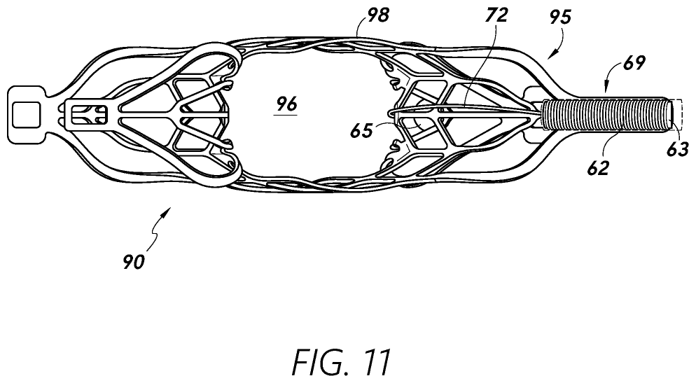

FIG. 11 illustrates another axial view of the implant device 60 of FIG. 9-1 in accordance with one or more embodiments of the present disclosure. Specifically, FIG. 11 shows an axial view that corresponds to an axial side of the implant device 60 associated with the antenna 69. That is, the antenna component 69 is attached to, integrated with, or otherwise associated with the arm 95, the side of which is shown facing out of the page in FIG. 11. The side shown facing out of the page in FIG. 11 may be a distal or proximal side.

The antenna component 69 can be mechanically attached or fastened to a portion of the arm 95. The antenna 69 may be attached to the anchor/arm 95 by any suitable or desirable attachment means, including adhesive attachment or mechanical engagement. For example, the arm 95 may comprise or be associated with one or more retention features, which may comprise one or more clamps, straps, ties, sutures, collars, clips, tabs, or the like. Such retention features may circumferentially encase or retain the antenna 69, or a portion thereof. In some embodiments, the antenna 69 may be attached to the arm 95 through the application of mechanical force, either through sliding the antenna 69 through certain retention features or through clipping, locking, or otherwise engaging the antenna 69 with the arm 95 by pressing or applying other mechanical force thereto. In some embodiments, the shunt structure 90 may comprise one or more tabs that may be configured to pop-up or extend on one or more sides of the antenna 69 for mechanical fastening. Such tabs may comprise memory metal (e.g., Nitinol) or other at least partially rigid material. In some embodiments, the antenna 69 is pre-attached to the arm 95 and/or integrated therewith prior to implantation. In some embodiments, the antenna 69 may be built or manufactured into the shunt structure 90 to form a unitary structure. For example, in some embodiments, the antenna 69 may be attached to or integrated with the arm member 95 of the shunt structure 90.

As described above, sensor-integrated shunt implant devices in accordance with embodiments of the present disclosure may be implanted in the wall separating the coronary sinus from the left atrium. FIG. 12 shows a sensor implant device 60 implanted in a tissue wall 21 between the coronary sinus 16 and the left atrium 2. The coronary sinus 16 is generally contiguous around the left atrium 2, and therefore there are a variety of possible acceptable placements for the implant device 60 and/or shunt structure 90. The target site selected for placement of the implant device 60 may be made in an area where the tissue of the particular patient is less thick or less dense, as determined beforehand by non-invasive diagnostic means, such as a CT scan or radiographic technique, such as fluoroscopy or intravascular coronary echo (IVUS).

As with other embodiments, the sensor implant device 60 includes a sensor assembly 61 including a sensor component 65, an antenna component 69, and a connector component 72 electrically and/or physically coupling the sensor component 65 to the antenna component 69. The sensor assembly 61 is disposed, attached, and/or otherwise secured or to or associated with the implant structure 90 (e.g., shunt structure) in a distributed manner as described in detail above. For example, the implant device 60 may be configured such that the sensor assembly 61 is attached thereto in a manner such that, when implanted, the sensor component 65 is at least partially exposed on the atrial side of the tissue wall 21, whereas the antenna component 69 is exposed/disposed at least partially on the coronary sinus side of the tissue wall 21, as shown. It should be understood, as represented by the dashed sensor assembly shown on the opposite radial side from the sensor assembly 61, that the sensor assembly 61 may be disposed on any radial/circumferential side/portion of the structure 90, such as on a side relatively close to the ostium of the coronary sinus and/or right atrium, or on a side oriented away from the coronary sinus ostium and/or right atrium.

The sensor implant device 60 can have two sensor assemblies attached to opposite radial sides of the shunt structure 90. In such embodiments, the sensor components (and antenna components) of the respective sensor assemblies may be disposed on the same axial side of the shunt structure 90 or on opposite axial sides. For example, including two sensor assemblies, wherein sensor components thereof are disposed on opposite axial sides of the shunt structure 90 can advantageously allow for measurement of physiological parameter(s) (e.g., pressure) in two chambers/vessels on either side of the tissue wall in which the device 60 is implanted. With pressure sensor functionality for measuring pressure in both chambers/vessels on either side of a tissue wall, the sensor implant device 60 may advantageously be configured to provide sensor signals that may be used to determine differential pressure across the chambers/vessels. In some embodiments, two sensor assemblies are implemented with the implant device 60, wherein the sensor assemblies comprise different types of sensor components for measuring different parameters. Such sensor components may be disposed on the same axial side of the structure 90 or on opposite sides.

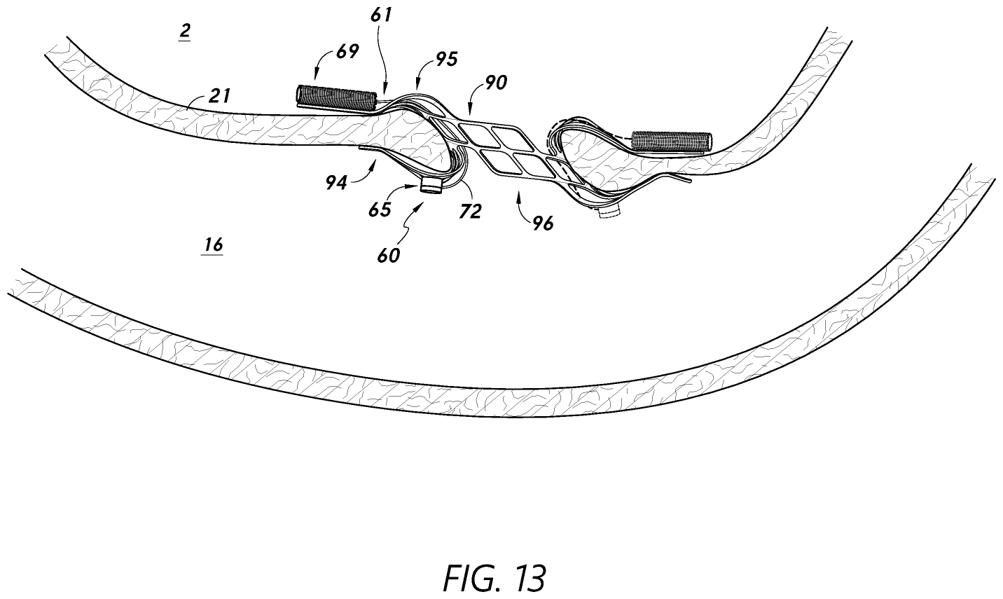

FIG. 13 shows the sensor implant device 60 implanted and configured in a manner such that the sensor component 65 is at least partially exposed on the coronary sinus side of the tissue wall 21, whereas the antenna component 69 is exposed/disposed at least partially on the atrial side of the tissue wall 21, as shown. It should be understood, as represented by the dashed sensor assembly shown on the opposite radial side from the sensor assembly 61, that the sensor assembly 61 may be disposed on any radial/circumferential side/portion of the structure 90, such as on a side relatively close to the ostium of the coronary sinus and/or right atrium, or on a side oriented away from the coronary sinus ostium and/or right atrium.

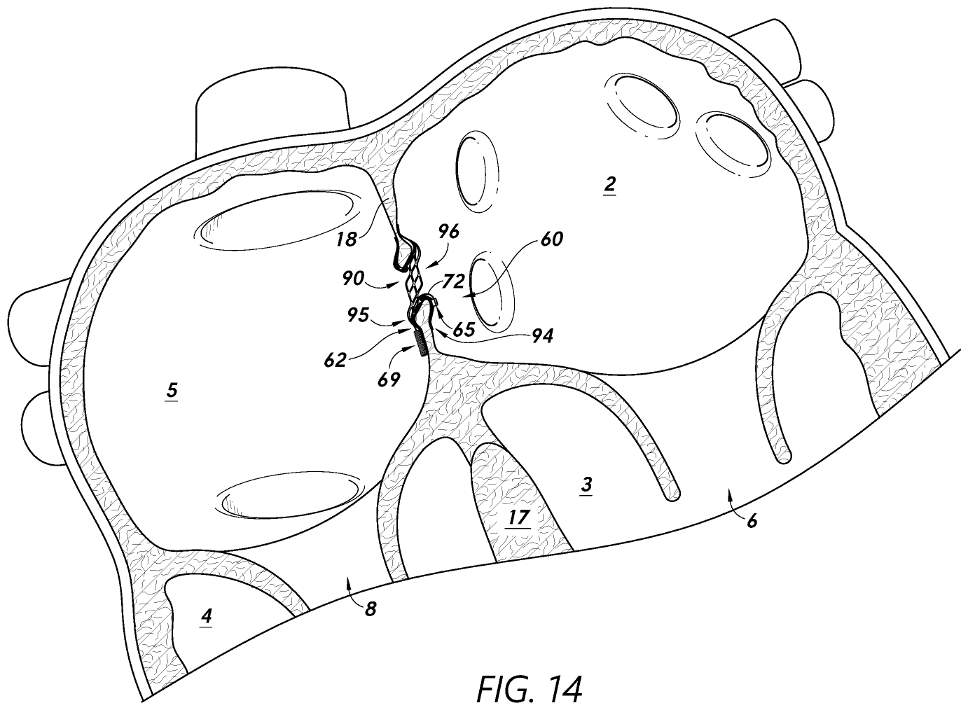

As described above, sensor-integrated shunt implant devices in accordance with embodiments of the present disclosure may be implanted in the interatrial septum. FIG. 14 shows a sensor implant device 60 implanted in a septal wall 18 between the left 2 and right 5 atria.

As with other embodiments, the sensor implant device 60 shown in FIG. 14 includes a sensor assembly 61 including a sensor component 65, an antenna component 69, and a connector component 72 electrically and/or physically coupling the sensor component 65 to the antenna component 69. The sensor assembly 61 is disposed, attached, and/or otherwise secured or to or associated with the implant structure 90 (e.g., shunt structure) in a distributed manner as described in detail above. For example, the implant device 60 may be configured with the sensor assembly 61 attached thereto in a manner such that, when implanted, the sensor component 65 is at least partially exposed/disposed on the left atrium side of the septal wall 18, whereas the antenna component 69 is exposed/disposed at least partially on the right atrium side of the tissue wall 21, as shown.

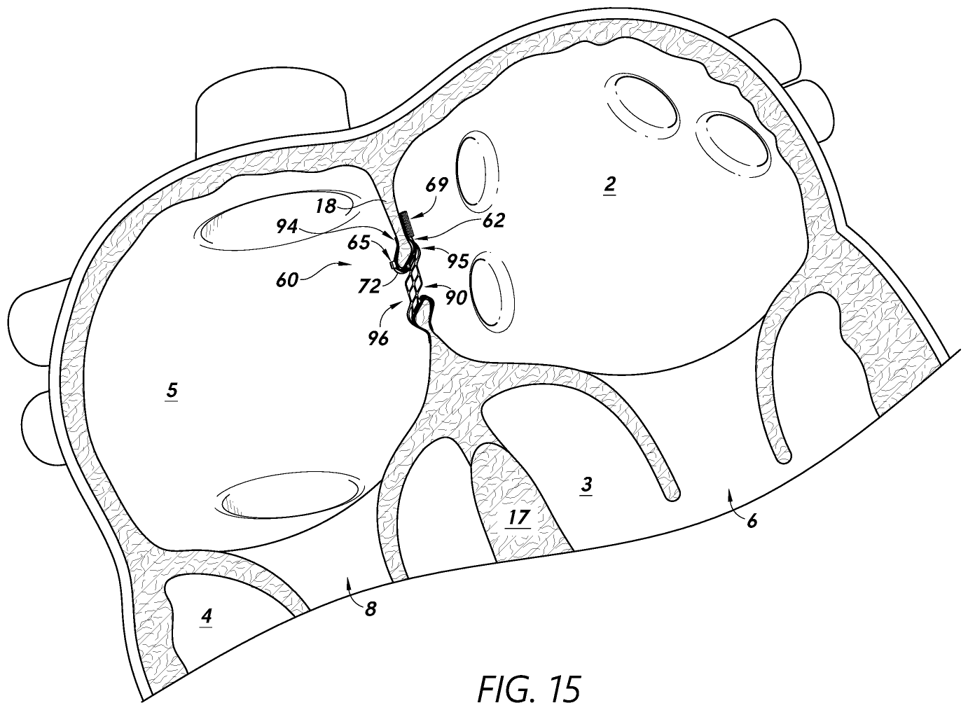

FIG. 15 shows the sensor implant device 60 implanted and configured in a manner such that the sensor component 65 is at least partially exposed/disposed on the right atrium side of the tissue wall 21, whereas the antenna component 69 is exposed/disposed at least partially on the left atrium side of the tissue wall 21, as shown.

The particular position in the interatrial septum wall 18 may be selected or determined in order to provide a relatively secure anchor location for the shunt structure 90, as well as to provide a relatively low risk of thrombus. Furthermore, the sensor implant device 60 may be implanted at a position that is desirable in consideration of future re-crossing of the septal wall 18 for future interventions. Implantation of the sensor implant device 60 in the interatrial septum wall may advantageously allow for fluid communication between the left 2 and right 5 atria. With the device 60 in the atrial septum 18, the sensor 65 of the sensor implant device 60 may advantageously be configured to measure pressure in the right atrium 5, the left atrium 2, or both atria. For example, in some embodiments, the device 60 comprises a plurality of sensors, wherein one sensor is disposed in each of the right atrium 5 and the left atrium 2. With pressure sensor functionality for measuring pressure in both atria, the sensor implant device 60 may advantageously be configured to provide sensor signals that may be used to determine differential pressure between the atria. Differential pressure determination may be useful for monitoring fluid build-up in the lungs, which may be associated with congestive heart failure.

Left-to-right shunting in connection with physiological parameter (e.g., pressure) sensing functionality, as achieved in accordance with any of the devices and/or implantations associated with FIGS. 12-15, may advantageously be well-suited for patients that are relatively highly sensitive to atrial pressure increases. For example, as pressure increases in the ventricles and/or atria and is applied against the myocardial cells, the muscles of the heart may generally be prone to contract relatively harder in order to process the excess blood. Therefore, as the ventricle dilates or stretches, for patients with compromised contractility of the ventricle, such patients may become more sensitive to higher pressures in the ventricle and/or atria because the heart may be unable to adequately respond or react thereto. Furthermore, increases in left-side (e.g., left atrial) pressure can result in dyspnea, and therefore reduction in left-side pressure to reduce dyspnea and/or reduce incidences of hospital readmission may be desirable through left-to-right shunting. For example, when the ventricle experiences dysfunction such that is unable to accommodate build-up in fluid pressure, such fluid may backup into the atria, thereby increasing atrial pressure. With respect to heart failure, minimization of left ventricular end-diastolic pressure may be paramount. Because left ventricular end-diastolic pressure can be related to left atrial pressure, backup of fluid in the atrium can cause backup of fluid in the lungs, thereby causing undesirable and/or dangerous fluid buildup in the lungs. Left-to-right shunting, such as using shunt devices in accordance with embodiments of the present disclosure, can divert extra fluid in the left side of the heart to the right side of the heart, which may be able to accommodate the additional fluid due to the relatively high compliance in the right atrium.

In some situations, left-to-right shunting may not be sufficiently effective due to the patient being subject to a drug regimen designed to control the patient's fluid output and/or pressure. For example, diuretic medications may be used to cause the patient to expel excess fluid. Therefore, use of pressure-sensor-integrated implants in accordance with embodiments of the present disclosure may provide a mechanism to inform technicians or doctors/surgeons with respect to how to titrate such medications to adjust/modify fluid status. Therefore, embodiments of the present disclosure may advantageously serve to direct medication intervention to reduce or prevent the undesirable increase in left atrial pressure.

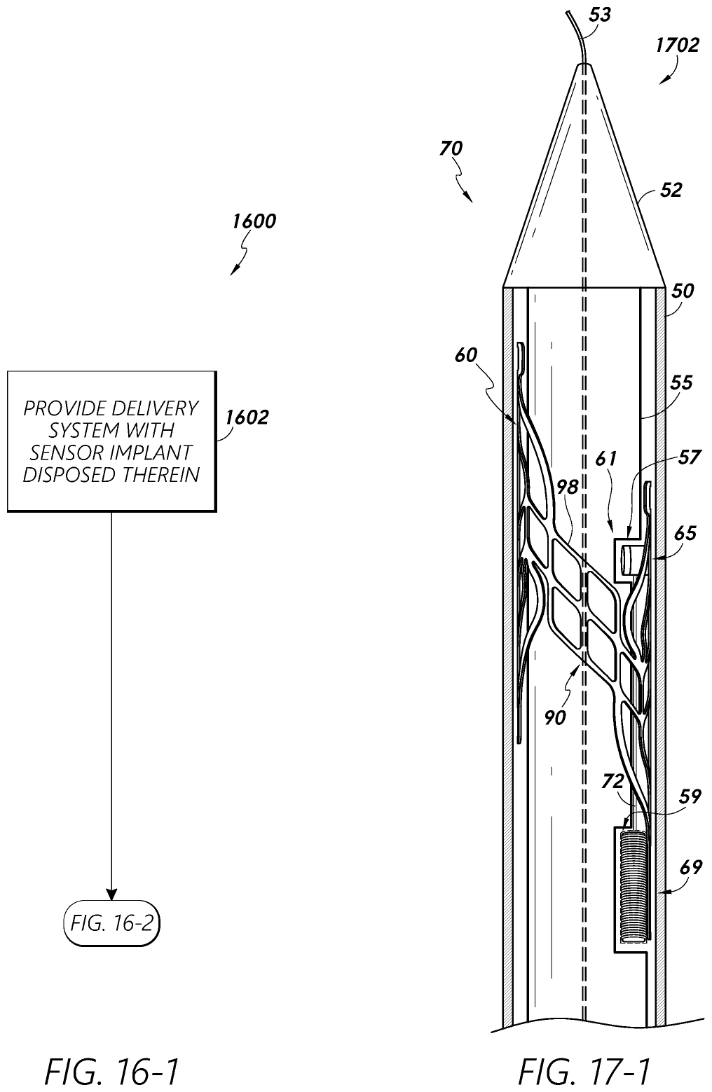

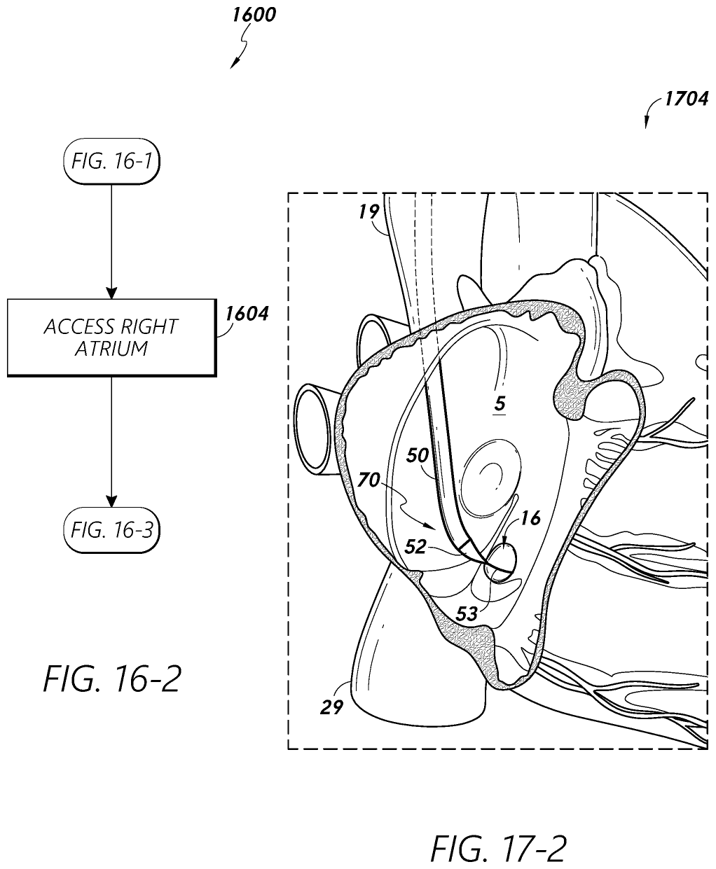

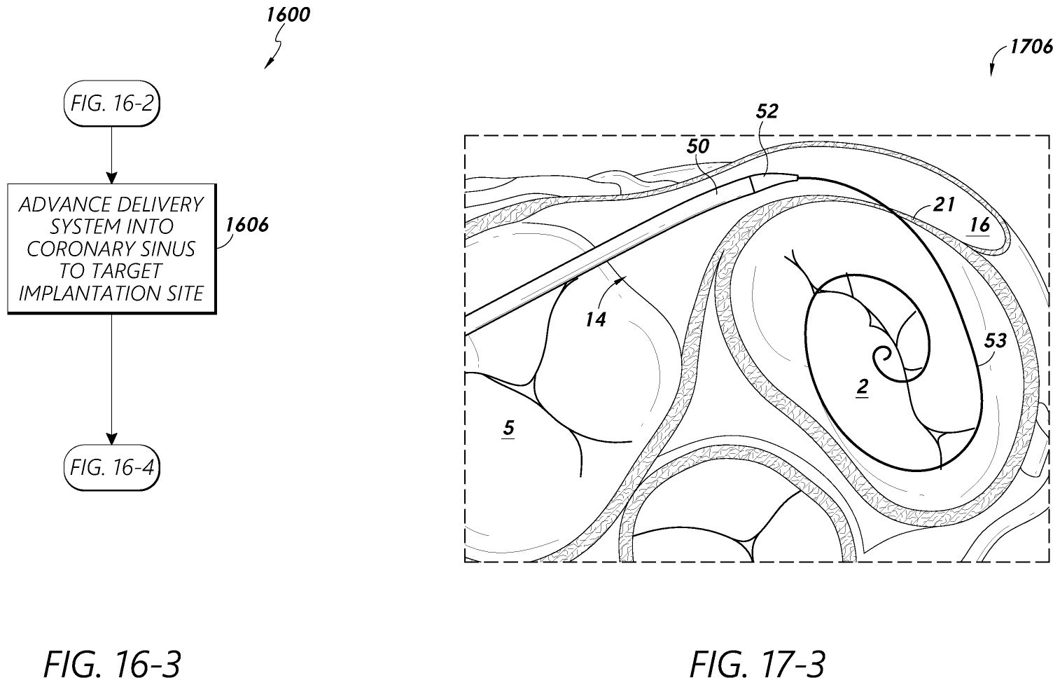

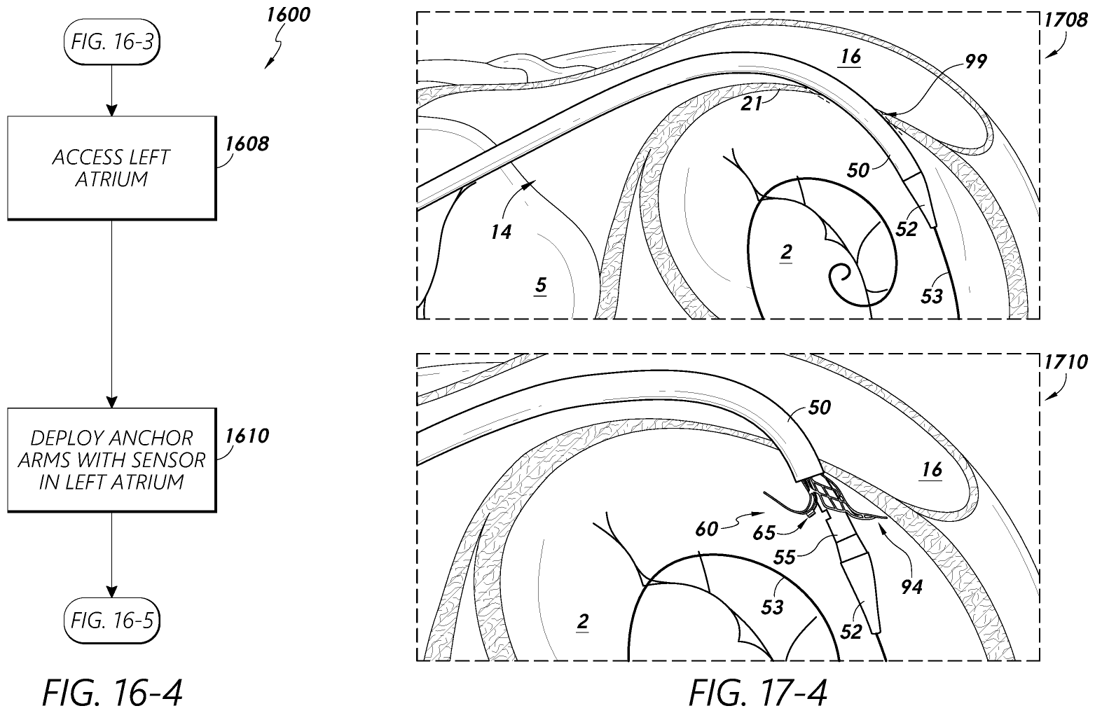

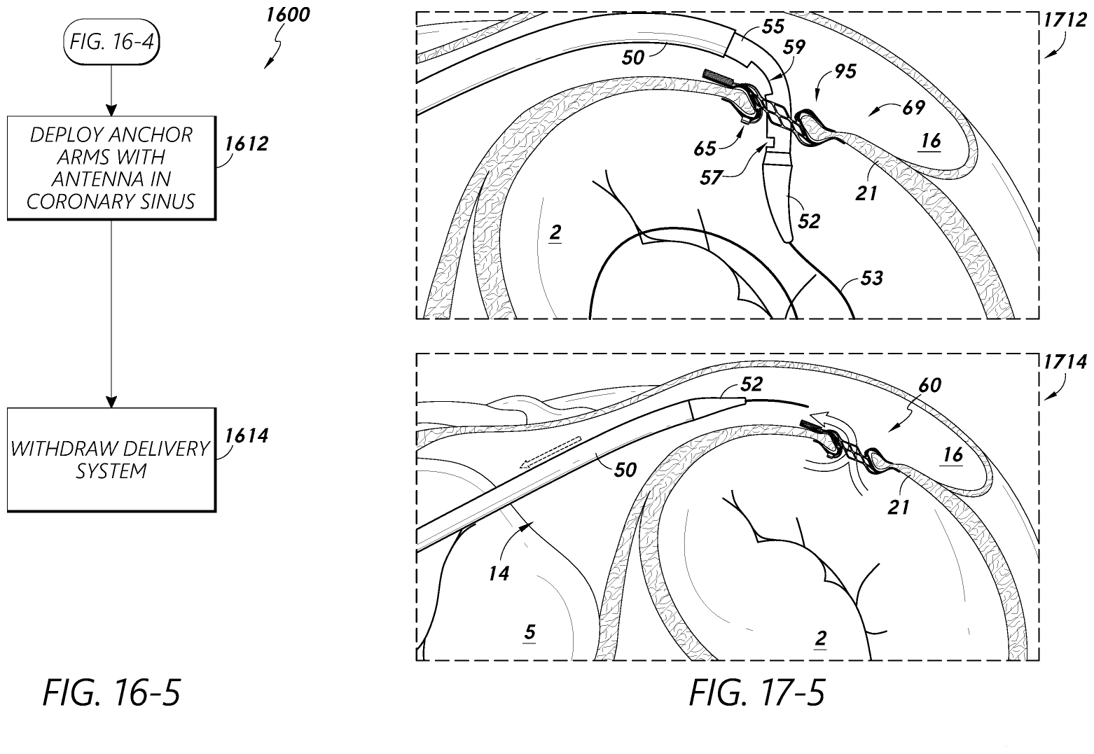

FIGS. 16-1, 16-2, 16-3, 16-4, and 16-5 provide a flow diagram illustrating a process 1600 for implanting a sensor implant device in accordance with one or more embodiments. FIGS. 17-1, 17-2, 17-3, 17-4, and 17-5 provide images of cardiac anatomy and certain devices/systems corresponding to operations of the process 1600 of FIGS. 16-1, 16-2, 16-3, 16-4, and 16-5 in accordance with one or more embodiments of the present disclosure.

At block 1602, the process 1600 involves providing a delivery system 70 with a sensor implant device 60 disposed therein in a delivery configuration, such as a shunt-type sensor implant device as disclosed in detail herein. Image 1702 of FIG. 17-1 shows a partial cross-sectional view of a delivery system 70 for a sensor implant device 60 in accordance with one or more embodiments of the present disclosure. The image 1702 shows the sensor implant device 60 disposed within an outer sheath 50 of the delivery system 70. Although a particular embodiment of a delivery system is shown in FIG. 17-1, it should be understood that sensor implant devices in accordance with aspects of the present disclosure may be delivered and/or implanted using any suitable or desirable delivery system and/or delivery system components.

The illustrated delivery system 70 includes an inner catheter 55, which may be disposed at least partially within the outer sheath 50 during one or more portions of the process 1600. In some embodiments, the shunt structure 90 of the sensor implant device 60 may be disposed at least partially around the inner catheter 55, wherein the shunt structure 90 is disposed at least partially within the outer sheath 50 during one or more portions of the process 1600. For example, the inner catheter 55 may be disposed within the barrel portion 98 of the shunt structure 90, as shown.

In some embodiments, the delivery system 70 may be configured such that a guidewire 53 may be disposed at least partially therein. For example, the guidewire 53 may run in the area of an axis of the sheath 50 and/or inner catheter 55, such as within the inner catheter 55, as shown. The delivery system 70 may be configured to be advanced over the guidewire 53 to guide the delivery system 70 to a target implantation site.

In some embodiments, the delivery system 70 includes a tapered nosecone feature 52, which may be associated with a distal end of the sheath 50, catheter 55, and/or delivery system 70. In some implementations, the nosecone feature 52 may be utilized to dilate the opening in a tissue wall into which the sensor implant device 60 is to be implanted, or through which the delivery system is to be advanced. The nosecone feature 52 may facilitate advancement of the distal end of the delivery system 70 through the tortuous anatomy of the patient and/or with an outer delivery sheath or other conduit/path. The nosecone 52 may be a separate component from the catheter 55 or may be integrated with the catheter 55. In some embodiments, the nosecone 52 is adjacent to and/or integrated with a distal end of the outer sheath 50. In some embodiments, the nosecone 52 may comprise and/or be formed of multiple flap-type forms that can be urged/spread apart when the sensor implant device 60 and/or any portions thereof, the interior catheter 55, or other device(s) are advanced therethrough.

In some embodiments, the sensor implant device 60 may be disposed in the delivery system 70 with a sensor assembly 61, as described in detail herein, attached thereto or otherwise associated therewith. In some embodiments, the inner catheter 55 includes one or more cut-outs, indentations, recesses, gaps, openings, apertures, holes, slits, or other features configured to accommodate the presence of the sensor component 65, the antenna component 69, the connector 72, and/or other feature(s) or aspect(s) of the sensor assembly 61. For example, the sensor assembly 61 may be disposed at least partially within an inner diameter of the shunt structure 90 in the delivery configuration shown in FIG. 17-1. In such configurations, the sensor assembly component(s) may create an interference with respect to the ability of the shunt structure 90 to be disposed relatively tightly around the inner catheter 55, thereby potentially increasing the profile of the delivery system and/or affecting the ability of the sensor implant device 60 to be delivered using the delivery system. Therefore, as shown in FIG. 17-1, the inner catheter 55 may include one or more sensor component accommodation features, such as a sensor cut-out or other accommodation feature 57 and/or an antenna cut-out or other accommodation feature 59. In some embodiments, the accommodation features 57, 59 may be longitudinal and circumferential cut-outs of the inner catheter 55. The accommodation features 57, 59 may advantageously be dimensioned to correspond to the size and/or profile of the respective sensor assembly components, as shown, and may allow for the sensor assembly components (e.g., sensor component 65 and/or antenna component 69) to radially project into an inner diameter/space of the inner catheter 55.

The implant sensor device 60 can be positioned within the delivery system 70 with a first end thereof (i.e., distal anchor arms) disposed distally with respect to the barrel 98 of the shunt structure 90 and/or one or more components of the sensor assembly 61. A second end (i.e., proximal anchor arms) is positioned at least partially proximally with respect to the barrel 98 of the shunt structure 90 and/or one or more components of the sensor assembly 61.

The outer sheath 50 may be used to transport the sensor implant device 60 to the target implantation site. That is, the sensor implant device 60 may be advanced to the target implantation site at least partially within a lumen of the outer sheath 50, such that the sensor implant device 60 is held and/or secured at least partially within a distal portion of the outer sheath 50.

At block 1604, the process 1600 involves accessing a right atrium of a heart of a patient using the delivery system 70 with the sensor implant device 60 disposed therein. In some implementations, accessing the cardiac anatomy with the delivery system 70 may be performed following one or more procedures or steps to place the guidewire 53 and form and/or dilate an opening between the left atrium and coronary sinus of the patient's heart, the details of which are omitted for convenience and clarity.

At block 1606, the process 1600 involves advancing the delivery system into the coronary sinus 16 to a target implantation site adjacent a wall 21 separating the coronary sinus 16 from the left atrium 2. Access to the target wall 21 and left atrium 2 via the coronary sinus 16 may be achieved using any suitable or desirable procedure. For example, various access pathways may be utilized in maneuvering guidewires and catheters in and around the heart to deploy an expandable shunt integrated or associated with a pressure sensor in accordance with embodiments of the present disclosure. In some embodiments, access may be achieved through the subclavian or jugular veins into the superior vena cava (not shown), right atrium 5, and from there into the coronary sinus 16. Alternatively, the access path may start in the femoral vein and through the inferior vena cava (not shown) into the heart. Other access routes may also be used, each of which may typically utilize a percutaneous incision through which the guidewire and catheter are inserted into the vasculature, normally through a sealed introducer, and from there the system may be designed or configured to allow the physician to control the distal ends of the devices from outside the body.

In some implementations, the guidewire 53 is introduced through the subclavian or jugular vein, through the superior vena cava 19, and into the coronary sinus 16 via the right atrium 5. The guidewire can be disposed in a spiral configuration within the left atrium 2, which may help to secure the guidewire in place. Once the guidewire 53 provides a path, an introducer sheath may be routed along the guidewire 53 and into the patient's vasculature, such as with the use of a dilator. The delivery catheter may be advanced through the superior vena cava to the coronary sinus of the heart, wherein the introducer sheath may provide a hemostatic valve to prevent blood loss. In some embodiments, a deployment catheter may function to form and prepare an opening in the wall of the left atrium, and a separate placement delivery system 70, as shown, is used for delivery of the sensor implant device 60. In other embodiments, the deployment system 70 may be used as the both the puncture preparation and implant delivery catheter with full functionality. In the present application, the term “delivery system” is used to represent a catheter or introducer with one or both of these functions.

At block 1608, the process 1600 involves accessing the left atrium through an opening 99 formed in the wall 21. For example, the guidewire 53 may be disposed as running through the opening 99 prior to penetration thereof by the nosecone 52. The opening 99 may originally be formed using a needle (not shown) associated with the delivery system 70 or other delivery system implemented prior to block 1608. In some implementations, the nosecone feature 52 may be used to at least partially dilate the opening 99, which may have been previously dilated using a balloon dilator or other instrument.

At block 1610, the process 1600 involves deploying one or more anchor arms 94, which may be considered the distal anchor arm (as) of the sensor implant device 60, on the atrial side of the wall 21. The distal arm 94 can have associated therewith a sensor 65 component of the sensor assembly 61, such that a sensor transducer of the sensor component 65 is exposed within the left atrium 2, such that the sensor component 65 can be used to obtain signals indicating physiological parameters associated with the left atrium, such as pressure.

At block 1612, the process 1600 involves deploying one or more proximal arms of the sensor implant device 60 on a coronary sinus side of the tissue wall 21 to thereby sandwich portions of the wall 21 between the distal and proximal arms of the shunt structure 90. One of the proximal arms 95 may advantageously have associated there with an antenna component 69 that is physically and/or electrically coupled/tethered to the sensor component 65 via a connector 72, as described in detail herein. At block 1614, the process 1600 involves withdrawing the delivery system leaving the sensor implant device 60 implanted in the tissue wall 21 thereby allowing blood flow to be shunted through the implant device 60 from the left atrium into the right side of the heart via the coronary sinus 16.

Additional aspects and features of processes for delivering shunt structures that may be integrated with sensor devices/functionality in accordance with embodiments of the present disclosure for implantation in the wall between the coronary sinus and the left atrium are disclosed in U.S. Pat. No. 9,789,294, entitled “Expandable Cardiac Shunt,” issued on Oct. 17, 2017, the disclosure of which is hereby expressly incorporated by reference in its entirety. Although the implant device 60 is shown in the left atrium/coronary sinus wall, the implant device 60 may be positioned between other cardiac chambers, such as between the left and right atria.

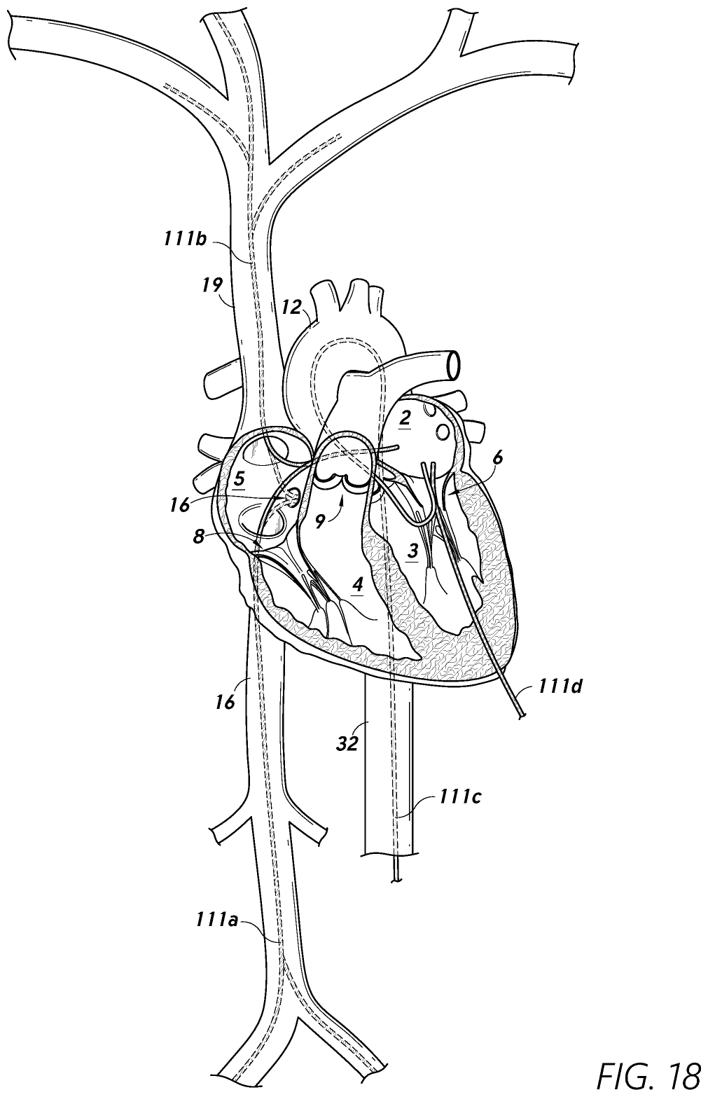

FIG. 18 is a cutaway view of a human heart and associated vasculature showing certain catheter access paths for implanting sensor implant devices in accordance with one or more embodiments. FIG. 18 shows various catheters 111 that may be used to implant sensor devices in accordance with aspects of the present disclosure. The catheters 111 can advantageously be steerable and relatively small in cross-sectional profile to allow for traversal of the various blood vessels and chambers through which they may be advanced en route to, for example, the right atrium 5, coronary sinus 16, left atrium 2 or other anatomy or chamber. Catheter access to the right atrium 5, coronary sinus 16, or left atrium 2 in accordance with certain transcatheter solutions may be made via the inferior vena cava 16 (as shown by the catheter 111a) or the superior vena cava 19 (as shown by the catheter 111b). Further access to the left atrium may involve crossing the atrial septum (e.g., in the area at or near the fossa ovalis).

Although access to the left atrium is illustrated and described in connection with certain examples as being via the right atrium and/or vena cavae, such as through a transfemoral or other transcatheter procedure, other access paths/methods may be implemented in accordance with examples of the present disclosure. For example, in cases in which septal crossing through the interatrial septal wall is not possible, other access routes may be taken to the left atrium 2. In patients suffering from a weakened and/or damaged interatrial septum, further engagement with the septal wall can be undesirable and result in further damage to the patient. Furthermore, in some patients, the septal wall may be occupied with one or more implant devices or other treatments, wherein it is not tenable to traverse the septal wall in view of such treatment(s). As alternatives to transseptal access, transaortic access may be implemented, wherein a delivery catheter 111c is passed through the descending aorta 32, aortic arch 12, ascending aorta, and aortic valve 7, and into the left atrium 2 through the mitral valve 6. Alternatively, transapical access may be implemented to access the target anatomy, as shown by delivery catheter 111d.

### ADDITIONAL EMBODIMENTS

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A sensor implant device comprising:
a shunt body that forms a fluid conduit;
a first anchor structure associated with a first end of the shunt body;
a second anchor structure associated with a second end of the shunt body;
a sensor device coupled to the first anchor structure; and
an antenna coupled to the second anchor structure.

2. The sensor implant device of claim 1, further comprising an electrical connector electrically connecting the sensor device to the antenna.

3. The sensor implant device of claim 2, wherein the electrical connector is disposed at least partially within the fluid conduit of the shunt body.

4. The sensor implant device of claim 1, wherein the antenna comprises a coil wrapped around a magnetic core.

5. The sensor implant device of claim 1, wherein the first anchor structure comprises one or more anchor arms configured to extend radially outward with respect to an axis of the fluid conduit, and the sensor device is coupled to one of the one or more anchor arms.

6. The sensor implant device of claim 5, wherein the sensor device includes a sensor transducer including a sensor membrane that faces in a direction within 30° of the axis of the fluid conduit.

7. The sensor implant device of claim 1, wherein the shunt body comprises a frame having a plurality of apertures therein.

8. The sensor implant device of claim 1, wherein the first anchor structure and the second anchor structure are configured to hold a portion of a tissue wall therebetween, and when the first anchor structure and the second anchor structure are holding the portion of the tissue wall, the portion of the tissue wall is disposed between the sensor device and the antenna.

9. The sensor implant device of claim 1, wherein, when the first anchor structure and the second anchor structure are projected radially outward with respect to an axis of the fluid conduit, the sensor device and the antenna are radially outside of an axial channel of the fluid conduit.

10. The sensor implant device of claim 9, wherein, when the first anchor structure and the second anchor structure are projected axially with respect to the axis of the fluid conduit in a delivery configuration of the sensor implant device, the sensor device and the antenna are within the axial channel of the fluid conduit.

11. A sensor implant device comprising:
a tubular frame;
a first anchor means associated with a first end of the tubular frame;
a second anchor means associated with a second end of the tubular frame;
a sensor device coupled to the first anchor means; and
a wireless transmitter means coupled to the second anchor means.

12. The sensor implant device of claim 11, further comprising a wire electrically connecting the sensor device to the wireless transmitter means.

13. The sensor implant device of claim 12, wherein the wire axially traverses the tubular frame.

14. The sensor implant device of claim 13, wherein the wire runs inside the tubular frame between the first end and the second end.

15. The sensor implant device of claim 11, wherein the wireless transmitter means comprises a conductive coil.

16. The sensor implant device of claim 15, wherein the conductive coil is wrapped around a cylindrical magnetic core.

17. The sensor implant device of claim 11, wherein:
the first anchor means comprises a first anchor arm configured to extend radially outward with respect to an axis of the tubular frame;
the sensor device is coupled to the first anchor arm;
the second anchor means comprises a second anchor arm configured to extend radially outward with respect to the axis of the tubular frame; and
the wireless transmitter means in coupled to the second anchor arm.

18. The sensor implant device of claim 11, wherein, when the first anchor means and the second anchor means are projected radially outward with respect to an axis of the tubular frame, the sensor device and the wireless transmitter means are radially outside of the tubular frame.

19. The sensor implant device of claim 18, wherein, when the first anchor means and the second anchor means are oriented axially with respect to the axis of the tubular frame, the sensor device and the wireless transmitter means are radially within the tubular frame.

20. A method of manufacturing a shunt implant device, the method comprising:
forming a shunt structure including a shunt body configured to form a tubular conduit, a first anchor structure associated with a first axial end of the shunt body, and a second anchor structure associated with a second axial end of the shunt body;
coupling a sensor device to the first anchor structure; and
coupling an antenna to the second anchor structure.
